(19) United States
(12) Patent Application Publication
(10) Pub. No.: US 2003/0082740 A1
(43) Pub. Date: May 1, 2003

(54) Histidine proline rich glycoprotein (HPRG) as an anti-angiogenic and anti-tumor agent

(76) Inventors: Fernando Donate, San Diego, CA (US); Scott Harris, San Diego, CA (US); Marian L. Plunkett, San Diego, CA (US); Andrew P. Mazar, San Diego, CA (US)

Correspondence Address: Venable, P.O. Box 34385, Washington, DC, 20043-9998, US

(21) Appl. No.: 10/074,225

(22) Filed: Feb. 14, 2002

Related U.S. Application Data
(60) Provisional application No. 60/268,370, filed on Feb. 14, 2001.

(51) Int. Cl. C12N 9/99; C07H 21/04; C12P 21/02; C12N 5/06

(52) U.S. Cl. 435/69.2; 435/184; 435/320.1; 435/325; 536/23.2

(57) ABSTRACT

Histidine Proline Rich Glycoprotein (HPRG) polypeptides or fragments thereof including pentapeptide fragments and multimers thereof, and other biologically active derivatives of HPRG are anti-angiogenic. These compounds may be used to inhibit angiogenesis or treat a disease or condition in which angiogenesis is pathogenic. These compounds therefore have anti-tumor activity and are used in methods for inhibiting the growth of primary tumors or metastases. Antibodies specific for epitopes of the His-Pro rich domain of HPRG are stimulators of angiogenesis and are useful for promoting neovascularization in pertinent disease states.

### BACKGROUND OF THE INVENTION

1. Field of the Invention

The present invention in the field of biochemistry and medicine is directed to novel methods for inhibiting angiogenesis and treating tumors and cancer using a glycoprotein termed “histidine proline rich glycoprotein” or biologically active fragments and other derivatives thereof.

2. Description of the Background Art

Angiogenesis, the formation of new capillaries form pre-existing ones (Folkman, J., N. Engl. J. Med., 1971, 285:1182-1186; Hanahan D. et al., Cell, 1996, 86:353-364), is a normal part of embryonic development, wound healing and female reproductive function. However, angiogenesis also plays a pathogenic role in the establishment and progression of certain diseases. Cancer, rheumatoid arthritis and diabetic retinopathy are examples of such diseases (Carmeliet P. et al., Nature, 2000, 407:249-257). Anti-angiogenic therapy holds promise in inhibiting the progression of these diseases.

Angiogenesis can be triggered by several pro-angiogenic cytokines. In the setting of cancer, tumor cells under hypoxic conditions secrete vascular endothelial growth factor (VEGF) and/or fibroblast growth factor (bFGF). These proteins diffuse and bind to specific receptors on endothelial cells (ECs) in the local vasculature, perturbing the balance of pro- and anti-angiogenic forces in favor of angiogenesis. As a consequence of binding these proteins, ECs are activated to (a) secrete enzymes that induce remodeling of the associated tissue matrix, and (b) change the patterns and levels of expression of adhesion molecules such as integrins. Following matrix degradation, ECs proliferate and migrate toward the hypoxic tumor, resulting in the generation and maturation of new blood vessels.

Interestingly, many anti-angiogenic factors result from the degradation of matrix proteins—i.e., are a result of the action of pro-angiogenic enzymes. Examples include endostatin, a fragment of collagen XIII (O'Reilly, M. S. et al., Cell 1997, 88:277-285); kringle 5 of plasminogen (O'Reilly, M. S. et al., Cell, 994, 79:315-328) and PEX, the C-terminus non-catalytic subunit of MMP-2 (Brooks P. C. et al., Cell, 1998, 92:391-400).

The concept has emerged that, due to the abundance of pro-angiogenic factors, these anti-angiogenic molecules are unable to overcome the pro-angiogenic balance in a primary tumor. However, since they are secreted into circulation, these anti-angiogenic molecules are capable of inhibiting angiogenesis at other locations where tumor cells may have begun to invade. Consequently, micro-metastases comprising these tumor cells at these new locations remain dormant. This hypothesis explains the puzzling observation made by surgeons many years ago: at various times after surgical removal of a primary tumor in a patient with no obvious metastatic disease, the patient returns with advanced metastatic disease.

Thus, clinical intervention by treatment with one or more of the anti-angiogenic factors could inhibit the angiogenic process and halt tumor growth as well as metastasis. Significant evidence in the literature (cited above) supports this notion.

Histidine Proline Rich Glycoprotein (HPRG=Histidine Rich Glycoprotein, HRG)

HPRG is synthesized in the liver (Morgan W. T., “Histidine-Rich Glycoprotein,” In: Encyclopedia of Molecular Medicine, 2001. This glycoprotein has an unusually high percentage of Pro and His residues (human HPRG has 525 residues, 66 are His and 65 are Pro) which is reflected in its name. HPRG contains two cystatin-like domains at the N-terminus, and a His-Pro rich domain—also referred to herein as the “H/P domain”—(148 residues in human HPRG, of which 42 are His and 31 are Pro) between two Pro-rich domains at the C-terminus. The C-terminal domain is tethered back to the N-terminal domain (as in kininogen) and contains all three N-linked oligosaccharides; its sequence has diverged from cystatin enough to have lost all of the protease inhibitor activity of cystatin. HPRG is quite abundant in plasma (1.5 μM, 125 μg/ml). Despite this, very little is known about the physiological roles of HPRG.

HPRG binds a large array of ligands that can be divided in three major groups:

(1) ligands belonging to the coagulation/fibrinolysis systems such as heparin, plasminogen, fibrinogen, vitronectin and thrombospondin;

(2) small ligands, such as heme and transition metal ions (zinc, copper and nickel), and

(3) cells such as T cells (Lamb-Wharton R. J. et al., Cellular. Immunol. 1993, 152:544-555; Olsen, H M et al., Immunology 1996, 88:198-206), macrophages and platelets.

Based on the foregoing, several hypotheses have been proposed for possible roles for HPRG in modulating coagulation and fibrinolysis, metal transport and regulation of the immune system. However, no hypothesis has yet been able to explain and integrate the apparent “promiscuity” of binding of this multidomain protein.

Some of the biological properties of HPRG depend on pH or metal binding. For instance, HPRG binding to heparin or to glycosaminoglycans (GAG) on the surface of ECs is dependent on low pH or abundant Zn⁺² or Cu⁺² (Borza D-B. et al., J. Biol. Chem., 1998, 273:5493-5499). Binding of Zn⁺² or Cu⁺² to the His-Pro-rich domain allows for subsequent binding to GAGs. Modest changes in pH of 0.25-0.50 units (from pH 7.4 of normal plasma), as may occur during hypoxia or ischemia, induce the protonation of the His residues of the H/P domain. Thus, pH and metal binding are exquisite regulators of HPRG activity.

HPRG binds plasminogen when in solution or when bound to GAG on the EC surface. This cell surface binding promotes activation of plasminogen to plasmin by tissue plasminogen activator (tPA) (Borza D-B. et al., J. Biol. Chem., 1997, 272:5718-5726), which is pro-angiogenic. The conserved C-terminal Lys is essential for the interaction with plasminogen as is the N-terminal domain. HPRG also binds to the γ-chains of fibrinogen. At pH 6.8, but not at pH 7.4, HPRG enhances polymerization of fibrin by thrombin.

Binding of chicken HPRG (cHPRG) to heparan sulfate proteoglycans has been shown to displace bFGF and αXFGF from those sites (Brown K. J. et al., Biochemistry, 1994, 33:13918.

While the effect of HPRG on angiogenesis has not been investigated, it was speculated that the abovementioned effect may promote or inhibit bFGF activity. Related to this property, cHPRG at concentrations of ≧80 μg/ml (approximately 1 μM) significantly inhibited FGF-stimulated and baseline endogenous DNA synthesis in fibroblasts (Brown et al. supra). Since baseline proliferation was also inhibited, the effect may not be specific for FGF-stimulated DNA synthesis. Rather, HPRG may regulate DNA synthesis regardless of the nature of the stimulus. However, Brown et al. did not examine the possible effects of HPRG on ECs and angiogenic processes.

Rabbit and human HPRG are very similar in composition and function. Optimal alignment of the two proteins showed 63.5% sequence identity and 68.6% homology (Borza D-B. et al., Biochemistry, 1996, 35:1925-1934). The highest homology is at the N- and C-termini. However, the apparent lower homology in the His-Pro rich domain is due to substitutions of Pro for His in the rabbit molecule. The human protein contains 15 repeats of the sequence HHPHG while the rabbit protein has 2 repeats of this sequence, 6 repeats of HPPHG and 7 repeats of PPPHG. Thus a consensus sequence for these repeating units is designated [H/P][H/P]PHG. Simantov, R. et al., J. Clin. Invest. 107:45-52 (2001) disclosed that HPRG inhibited the anti-angiogenic activity of thrombospondin (TSP-1) and concluded that regions of the HPRG are homologous to CD36, a TSP-1 receptor. These regions are at the N-terminus of HPRG, which contrasts from the present inventors' localization of anti-angiogenic activity to the H/P domain

### SUMMARY OF THE INVENTION

The present inventors have discovered that HPRG polypeptides or fragments thereof including domains and pentapeptides, altered conformations of HPRG, other biologically active derivatives of HPRG, exhibit anti-angiogenic and anti-tumor activity whereas antibodies specific for HPRG stimulate angiogenesis by blocking the action of HPRG in vivo. The anti-angiogenic action may occur in part through inhibition of oxidative stress, which has recently been demonstrated in vitro to contribute to the pathophysiology of angiogenesis (Brown et al. (2000) Cancer Res. 60:6298). Oxidative stress leading to angiogenesis may require transition metals such as zinc and copper—small molecule copper chelators have been demonstrated to inhibit tumor growth in vivo (Brewer, G J, International Patent publication WO/013712 (2000)).

The present invention includes the first demonstration that proteinaceous metal chelator (HPRG) inhibits angiogenesis, possibly due to its binding transition metals. The present invention provides novel methods to inhibit or reduce angiogenesis, tumor growth, EC proliferation, EC migration or EC tube formation using HPRG, domains and peptide fragments, altered conformations and other biologically active derivatives.

Transition metals and induction of oxidative stress have been implicated in the etiology of non-cancerous diseases, especially, neurodegenerative diseases such as Alzheimer's disease, Parkinson's disease and amyotrophic lateral sclerosis (ALS). Thus, the present invention also provides compositions and methods for the treatment of any disease whose pathobiology involves abnormal presence or undesired action of transition metals, including conditions where the presence of the transition metal may induce oxidative stress.

The present invention provides an isolated anti-angiogenic polypeptide or peptide having the sequence of

(a) the histidine-proline-rich (H/P) domain of human histidine-proline rich glycoprotein (HPRG) (SEQ ID NO:5)

(b) the H/P domain of human rabbit HPRG (SEQ ID NO:6)

(c) a sequence variant of SEQ ID NO:5 or SEQ ID NO:6 having substantially the same biologic activity of inhibiting angiogenesis, endothelial cell proliferation or endothelial tube formation in an in vitro or in vivo bioassay;

(d) a pentapeptide from the H/P domain having the sequence (His,Pro)-(His,Pro)-Pro-His-Gly (SEQ ID NO:7), or an addition variant thereof having an additional 1 to 4 amino acids selected from the group consisting of His, Pro or Gly added at the N- or C-terminus of the pentapeptide.

The isolated peptide above preferably has a sequence selected from the group consisting of His-His-Pro-His-Gly (SEQ ID NO:8), His-Pro-Pro-His-Gly (SEQ ID NO:9), or Pro-Pro-Pro-His-Gly (SEQ ID NO:10).

Also provided is a chemically synthesized peptide multimer comprising the above peptide or addition variant, which multimer is selected from the group consisting of:

(a) a multimer having the formula P¹ₙ wherein

(i) P¹ is the peptide or addition variant of claim 2, and

(ii) n=2-8,

(b) a multimer having the formula (P¹-Xₘ)ₙ-P², wherein

(i) P¹ and P² are pentapeptides or addition variants according to claim,

(ii) P¹ and P² are the same or different peptides;

(iii) X is C₁-C₅ alkyl, C₁-C₅ alkenyl, C₁-C₅ alkynyl, C₁-C₅ polyether containing up to 4 oxygen atoms,

(iv) m=0 or 1 and

(v) n 1-7

and wherein the peptide multimer has the biological activity of inhibiting angiogenesis, endothelial cell proliferation or endothelial tube formation in an in vitro or in vivo bioassay.

Another embodiment is a recombinantly produced peptide multimer comprising the above peptide or addition variant, which multimer has the formula (P¹-Gly_{z})ₙ-P², wherein:

(i) P¹ and P²are pentapeptides or addition variants according to claim 2,

(ii) P¹ and P²ₐᵣₑ the same or different;

(iii) z=0-6; and

(iv) n=1-100.

The present invention is also directed to a diagnostically or therapeutically labeled anti-angiogenic polypeptide, peptide or peptide multimer comprising:

(a) the polypeptide, peptide or peptide multimer above, which is diagnostically or therapeutically labeled;

(b) a diagnostically or therapeutically labeled human HPRG protein (SEQ ID NO:1);

(c) a diagnostically or therapeutically labeled rabbit HPRG protein (SEQ ID NO:3); or

(d) a diagnostically or therapeutically labeled polypeptide that is a homologue of (b) or (c).,

Preferably, the diagnostically or therapeutically labeled polypeptide or peptide is selected from the group consisting of: (a) the H/P domain of human HPRG (SEQ ID NO:5); (b) the H/P domain of rabbit HPRG (SEQ ID NO:6); and (c) the peptide having the sequence SEQ ID NO:7 or the addition variant thereof.

A diagnostically useful HPRG-related composition comprises the diagnostically labeled protein, peptide or peptide multimer as above, and a diagnostically acceptable carrier.

In the above diagnostic composition the detectable label is preferably a radionuclide, a PET-imageable agent, an MRI-imageable agent, a fluorescer, a fluorogen, a chromophore, a chromogen, a phosphorescer, a chemiluminescer or a bioluminescer.

Preferred radionuclides include ³H, ¹⁴C, ³⁵S, ⁶⁷Ga, ⁶⁸Ga, ⁷²As, ⁸⁹Zr, ⁹⁷Ru, ⁹⁹Tc, ¹¹¹In, ¹²³I, ¹²⁵I, ¹³¹I, ¹⁶⁹Yb and ²⁰¹Tl.

Preferred fluorescers or fluorogens include fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green and Texas Red.

An anti-angiogenic pharmaceutical composition comprises an effective amount of the protein peptide or peptide multimer of any of claims 1-4; and a pharmaceutically acceptable carrier.

In one embodiment, a therapeutic anti-angiogenic pharmaceutical composition comprises an effective amount of the polypeptide, peptide or peptide multimer described above to which is bound directly or indirectly a therapeutically active moiety; and a pharmaceutically acceptable carrier. Preferably the pharmaceutical composition is in a form suitable for injection.

The therapeutically active moiety may be a radionuclide, preferably ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb or ²¹⁷Bi.

This invention is also directed to an antibody specific for an epitope of HPRG that is present in the H/P domain of human HPRG (SEQ ID NO:5) or the H/P domain of rabbit HPRG (SEQ ID NO:6), and which binds to HPRG or to any of the domains in a way which inhibits the anti-angiogenic activity of HPRG or the domain, (or an antigen-binding fragment of the antibody). The epitope recognized by the antibody or fragment preferably comprises a pentapeptide from the H/P domain having the sequence His-His-Pro-His-Gly (SEQ ID NO:8), His-Pro-Pro-His-Gly (SEQ ID NO:9), or Pro-Pro-Pro-His-Gly (SEQ ID NO:10). The antibody may be a monoclonal antibody, including a human or humanized monoclonal antibody.

An antibody embodiment useful for detecting HPRG comprises the above antibody or fragment which is detectably labeled.

A therapeutically useful antibody that targets HPRG or an epitope thereof comprises the above antibody or fragment to which is bound directly or indirectly a therapeutically active moiety.

The invention provides a pharmaceutical composition that stimulates angiogenesis in vitro or in vivo, comprising: (a) the antibody or fragment above; and (b) a pharmaceutically acceptable carrier.

This invention provides a method for inhibiting cell migration, cell invasion, cell proliferation or angiogenesis, or for inducing apoptosis, comprising contacting cells associated with undesired cell migration, invasion, proliferation or angiogenesis with an effective amount of a therapeutic pharmaceutical composition as described above.

Also included is a method for treating a subject having a disease or condition associated with undesired cell migration, invasion, proliferation, or angiogenesis, comprising administering to the subject an effective amount of the pharmaceutical composition comprising the polypeptide, peptide or multimer. A preferred disease or condition for this treatment is a tumor or cancer.

Another method for stimulating angiogenesis comprises providing to cells participating in angiogenesis an effective amount of the antibody or fragment above. A method for stimulating angiogenesis in a subject in need of enhanced angiogenesis comprises administering to the subject an effective amount of the above antibody-based pharmaceutical composition.

Also provides is a method for detecting the presence of HPRG or cleavage product or peptide thereof in a biological sample, comprising the steps of:

(a) contacting the sample with the antibody or fragment of claim 20; and

(b) detecting the presence of the label associated with the sample.

The sample is preferably plasma, serum, cells, a tissue, an organ, or an extract of the cells, tissue or organ. The contacting and the detecting may be in vitro; alternatively, the contacting is in vivo and the detecting is in vitro or vice versa. In another embodiment, the contacting and the detecting are in vivo The present invention is also directed to an isolated nucleic acid that encodes the polypeptide or peptide or peptide multimer described above. An expression vector of this invention comprises the above nucleic acid of claim operatively linked to a promoter and optionally, additional regulatory sequences that regulate expression of the nucleic acid in a eukaryotic cell. A preferred expression vector is a plasmid or a viral vector.

Also included is a cell transformed or transfected with the above nucleic acid molecule or expression vector. The cell is preferably a mammalian cell, most preferably a human cell.

The invention includes a method for providing to a cell, tissue or organ an angiogenesis-inhibitory amount of a HPRG, an H/P domain of HPRG or a pentapeptide of the H/P domain having the sequence (His,Pro)-(His,Pro)-Pro-His-Gly (SEQ ID NO:7), or a peptide multimer that includes the pentapeptide, comprising: administering to the cell tissue or organ, the above expression vector such that the nucleic acid is taken up and expressed in the cell, tissue or organ. The administering is preferably in vivo.

Also included is a method for providing to a cell, tissue or organ an angiogenesis-inhibitory amount of a HPRG, an H/P domain of HPRG, a pentapeptide of the H/P domain having the sequence (His,Pro)-(His,Pro)-Pro-His-Gly (SEQ ID NO:7), or a peptide multimer that includes the pentapeptide, comprising: contacting the cell tissue or organ, with the above transformed or transfected cells, wherein the administered cells express the polypeptide, peptide or peptide multimer. Preferably, the contacting is in vivo.

This invention is also directed to a method for inhibiting angiogenesis in a subject in need of such inhibition, comprising administering to the subject an effective amount of the expression vector as above, such that the nucleic acid is expressed resulting in the presence of an angiogenesis-inhibiting amount of the polypeptide, peptide or peptide multimer, thereby inhibiting the angiogenesis.

Another method for inhibiting angiogenesis in a subject in need of such inhibition, comprises administering to the subject an effective amount of the transformed or transfected cells as above, which cells produce and provide in the subject an angiogenesis-inhibiting amount of the polypeptide, peptide or peptide multimer, thereby inhibiting the angiogenesis.

In the above methods, the subject has a tumor, and the angiogenesis inhibition results in reduction in size or growth rate of the tumor or destruction of the tumor. Preferably, the subject is a human.

A longer example of a disease or condition against which the above method is effective include primary growth of a solid tumor, leukemia or lymphoma; tumor invasion, metastasis or growth of tumor metastases; benign hyperplasia; atherosclerosis; myocardial angiogenesis; post-balloon angioplasty vascular restenosis; neointima formation following vascular trauma; vascular graft restenosis; coronary collateral formation; deep venous thrombosis; ischemic limb angiogenesis; telangiectasia; pyogenic granuloma; comeal disease; rubeosis; neovascular glaucoma; diabetic and other retinopathy; retrolental fibroplasia; diabetic neovascularization; macular degeneration; endometriosis; arthritis; fibrosis associated with a chronic inflammatory condition, traumatic spinal cord injury including ischemia, scarring or fibrosis; lung fibrosis, chemotherapy-induced fibrosis; wound healing with scarring and fibrosis; peptic ulcers; a bone fracture; keloids; or a disorder of vasculogenesis, hematopoiesis, ovulation, menstruation, pregnancy or placentation associated with pathogenic cell invasion or with angiogenesis.

A preferred disease or condition to be treated by the above method is tumor growth, invasion or metastasis. This in includes brain tumors. Examples of such brain tumors are astrocytoma, anaplastic astrocytoma, glioblastoma, glioblastoma multiformae, pilocytic astrocytoma, pleiomorphic xanthoastrocytoma, subependymal giant cell astrocytoma, fibrillary astrocytoma, gemistocytic astrocytoma, protoplasmic astrocytoma, oligodendroglioma, anaplastic oligodendroglioma, ependymoma, anaplastic ependymoma, myxopapillary ependymoma, subependymoma, mixed oligoastrocytoma and malignant oligoastrocytoma.

The method is also used to treat a uterine disease such as endometriosis and pathogenic ocular neovascularization such as that associated with, or a cause of, proliferative diabetic retinopathy, neovascular age-related macular degeneration, retinopathy of prematurity, sickle cell retinopathy or retinal vein occlusion.

Also provided herein is an “HPRG affinity ligand” useful for binding to or isolating HPRG-ligands, binding sites or cells expressing the ligands or binding sites, comprising the above polypeptides, peptide or peptide multimers immobilized to a solid support or carrier. This affinity ligand is used in a method for isolating a HPRG protein or peptide from a complex mixture comprising:

(a) contacting the mixture with the affinity ligand above;

(b) allowing any material in the mixture to bind to said ligand;

(c) removing unbound material from said ligand; and

(d) eluting the bound HPRG protein or peptide.

Also provided is a method for isolating or enriching cells expressing a HPRG binding site/receptor from a cell mixture, comprising

(a) contacting said cell mixture with the above HPRG affinity ligand;

(b) allowing any cells expressing the binding site to bind to said compound;

(c) separating cells bound to said compound from unbound cells; and

(d) removing said bound cells,

thereby isolating or enriching said HPRG binding site-expressing cells.

### BRIEF DESCRIPTION OF THE DRAWINGS

FIG. 1 is a schematic diagram of the structure of HPRG, showing the various domains. The scissors indicate the position of plasmin cleavage sites.

FIGS. 2A and 2B show inhibition of bFGF-stimulated proliferation of human umbilical vein endothelial cells (HUVEC). Rabbit HPRG (FIG. 2A) and its His-Pro rich (“H/P”) domain inhibit proliferation of HUVEC

FIG. 3 shows the induction of caspase-3 in bFGF-stimulated HUVEC by HPRG and HKa, the two-chain human kininogen protein.

FIGS. 4A and 4B are photomicrographs of HUVEC plated on Matrigel®-coated 96 well plates showing the inhibition of EC tube formation by HPRG (FIG. 4B) compared to a control (FIG. 4A).

FIG. 5 shows the inhibition of angiogenesis in the chorioallantoic membrane (CAM) using chick embryos. HPRG (ATN-234) and the H/P domain (ATN-236) are shown to inhibit angiogenesis, expressed as blood vessel number.

FIG. 6 shows that HPRG and the H/P domain inhibit angiogenesis stimulated by FGF-2 in Matrigel® plug model in vivo.

FIG. 7 shows that HPRG and the H/P domain inhibit 3LL tumor-mediated angiogenesis in Matrigel® plug model in vivo.

FIGS. 8A and 8B show that the H/P domain of HPRG inhibits growth of (FIG. 8A) and angiogenesis by (FIG. 8B) MatLyLu tumor cells in vivo in a Matrigel® Plug model. The H/P domain was tested at 1.8 μM (as was the positive control endostatin protein).

### DESCRIPTION OF THE PREFERRED EMBODIMENTS

No role for HPRG as an inhibitor of angiogenesis had been suggested prior to the making of the present invention. The present inventors conceived that native HPRG and biologically active HPRG polypeptides, homologues, variants and other functional derivatives including peptide fragments and conformers of HPRG, as well as antibodies specific for HPRG exhibit anti-angiogenic activity and, therefore, anti-tumor activity. Pharmaceutical compositions comprising these compounds are useful in the treatment of cancer and other diseases associated with aberrant or undesired angiogenesis.

Human HPRG has the amino acid sequence:

SEQ ID NO:1

```
                10 20 30 40
                  | | | |
MKALIAALLL ITLQYSCAVS PTDCSAVEPE AEKALDLINK
                50 60 70 80
                  | | | |
RRRDGYLFQL LRIADAHLDR VENTTVYYLV LDVQESDCSV
                90 100 110 120
                  | | | |
LSRKYWNDCE PPDSRRPSEI VIGQCKVIAT RHSHESQDLR
              130 140 150 160
                  | | | |
VIDFNCTTSS VSSALANTKD SPVLIDFFED TERYRKQANK
              170 180 190 200
                  | | | |
ALEKYKEEND DFASFRVDRI ERVARVRGGE GTGYFVDFSV
              210 220 230 240
                  | | | |
RNCPRHHFPR HPNVFGFCRA DLFYDVEALD LESPKNLVIN
              250 260 270 280
                  | | | |
CEVFDPQEHE NINGVPPHLG HPFHWGGHER SSTTKPPFKP
              290 300 310 320
                  | | | |
HGSRDHHHPH KPHEHGPPPP PDERDHSHGP PLPQGPPPLL
              330 340 350 360
                  | | | |
PMSCSSCQHA TFGTNGAQRH SHNNNSSDLH PHGHHSHEQH
              370 380 390 400
                  | | | |
PHGHHPHAHH PHEHDTHRQH PHGHHPHGHH PHGHHPHGHH
              410 420 430 440
                  | | | |
PHGHHPHCHD FQDYGPCDPP PHNQGHCCHG HGPPPGHLRR
              450 460 470 480
                  | | | |
RGPGKGPRPF HCRQIGSVYR LPPLRKGEVL PLPEANFPSF
              490 500 510 520
                  | | | |
PLPHHKHPLK PDNQPFPQSV SESCPGKFKS GFPQVSMFFT
    525
        |
HTFPK
```

italic: signal sequence

double underscore: Pro-rich domain

single underscore: His-Pro (H/P) rich domain

Thus, human HPRG consists of 525 amino acids residues, has a molecular mass of weight: 59,578 Da and a theoretical pI of 7.09

Human HPRG is encoded by the DNA of the following
sequence

4 (SEQ ID NO:2)

| | |
|---|---|
| 1 | atataatata aactaataaa gatcaggaaa taattaatgt |

ataccgtaat gtagaccgac

| | |
|---|---|
| 61 | tcaggtatgt aagtagagaa tatgaaggtg aattagataa |

ttaaagggat ggtttaacaa

| | |
|---|---|
| 121 | aatgaaggca ctcattgcag cactgctttt gatcacattg |

cagtattcgt gtgccgtgag

| | |
|---|---|
| 181 | tcccactgac tgcagtgctg ttgagccgga ggctgagaaa |

gctctagacc tgatcaacaa

| | |
|---|---|
| 241 | aaggcgacgg gatggctacc ttttccaatt gctgcggatt |

gctgatgccc acttggacag

| | |
|---|---|
| 301 | agtggaaaat acaactgtat attacttagt cttagatgtg |

caagaatcgg actgttcggt

| | |
|---|---|
| 361 | cctatccagg aaatactgga atgactgtga gccacctgat |

tccagacgtc catctgaaat

| | |
|---|---|
| 421 | agtgatcgga caatgtaagg taatagctac aagacattcc |

catgaatctc aggacctcag

| | |
|---|---|
| 481 | agtgattgac tttaactgca ccacaagttc tgtctcttca |

gcactggcca ataccaaaga

| | |
|---|---|
| 541 | tagtccggtc ctcatagatt tctttgagga tactgagcgc |

tacagaaaac aagccaacaa

| | |
|---|---|
| 601 | agcccttgag aagtacaaag aggagaatga tgactttgcc |

tctttcagag tggaccgaat

| | |
|---|---|
| 661 | cgagagagtt gcaagagtga gaggagggga aggaactggt |

tacttcgtgg acttctctgt

| | |
|---|---|
| 721 | gcggaactgc cccagacacc atttccccag acaccccaat |

gtctttggat tctgcagagc

| | |
|---|---|
| 781 | agatttgttc tatgatgtag aagccttgga cttggaaagc |

ccgaaaaacc ttgtcataaa

| | |
|---|---|
| 841 | ctgtgaagtc ttcgaccctc aggaacatga gaacatcaat |

ggtgtaccgc ctcatttggg

| | |
|---|---|
| 901 | acatcccttc cactggggtg ggcatgagcg ttcttctacc |

accaagcctc cattcaagcc

| | |
|---|---|
| 961 | ccatggatct agagatcatc atcatcccca caagccacac |

gaacatggac ccccacctcc

| | |
|---|---|
| 1021 | tccagatgaa agagatcact cacatggacc cccacttcca |

caaggccctc ctccactatt

| | |
|---|---|
| 1081 | gcccatgtcc tgctcaagtt gtcaacatgc cacttttggc |

acaaatgggg cccaaagaca

| | |
|---|---|
| 1141 | ttctcataat aataattcca gtgacctcca tccccataag |

catcattccc atgaacagca

| | |
|---|---|
| 1201 | tccccacgga caccatcccc atgcacacca tcctcatgaa |

catgataccc atagacagca

| | |
|---|---|
| 1261 | tccccatgga caccaccccc atggacacca tcctcatgga |

caccaccccc atggacacca

| | |
|---|---|
| 1321 | tccccatgga caccatcccc actgccatga tttccaagac |

tatggacctt gtgacccacc

| | |
|---|---|
| 1381 | accccataac caaggtcact gttgccatgg ccacggccca |

ccacctgggc acttaagaag

| | |
|---|---|
| 1441 | gcgaggccca ggtaaaggac cccgtccctt ccattgcaga |

caaattggat ctgtgtaccg

| | |
|---|---|
| 1501 | actccctcct ctaagaaaag gtgaggtgct gccacttcct |

gaggccaatt ttcccagctt

| | |
|---|---|
| 1561 | cccattgccg caccacaaac atcctctaaa gccagacaat |

cagccctttc ctcaatcagt

| | |
|---|---|
| 1621 | ctctgaatca tgtccaggga agttcaagag tgggtttcca |

caagtttcca tgttttttac

| | |
|---|---|
| 1681 | acatacattt ccaaaataaa atgtgattcc tttgaagagg |

aaaatgaata atacattgaa

| | |
|---|---|
| 1741 | ttagaaacat aaataaaatg accagtaatt gtgaaaatta |

cagttctttt caacctactt

| | |
|---|---|
| 1801 | tcatactgaa gatgcagcaa aatgtgaatg ggaaaagaga |

tggcctgaga agagagatca

| | |
|---|---|
| 1861 | aatggaaagg agaggaaaga actcagtgct gcctattagt |

agttaattct gtcactcacc

| | |
|---|---|
| 1921 | actacatcac ttgagacaaa tctatgccac tcagaatctc |

cttctttcct ggacttaact

| | |
|---|---|
| 1981 | ctaattctag agtctctgtt actgcttggg ctatacctgg |

gcatactaat aaagtatggt

| | |
|---|---|
| 2041 | attgaaacta t 2051 |

Rabbit HPRG has the amino acid sequence SEQ ID NO:
3 as follows:

```
                10 20 30 40
                  | | | |
ATLQCSWALT PTDCKTTKPL AEKALDLINK WRRDGYLFQL
                50 60 70 80
                  | | | |
LRVADAHLDG AESATVYYLV LDVKETDCSV LSRKHWEDCD
                90 100 110 120
                  | | | |
PDLTKRPSLD VIGQCKVIAT RYSDEYQTLR LNDFNCTTSS
              130 140 150 160
                  | | | |
VSSALANTKD SPVLFDFIED TEPFRKSADK ALEVYKSESE
              170 180 190 200
                  | | | |
AYASFRVDRV ERVTRVKGGE RTNYYVDFSV RNCSRSHFHR
              210 220 230 240
                  | | | |
HPAFGFCRAD LSFDVEASNL ENPEDVIISC EVFNFEEHGN
              250 260 270 280
                  | | | |
ISGFRPHLGK TPLGTDGSRD HHHPHKPHKF GCPPPQEGED
              290 300 310 320
                  | | | |
FSEGPPLQGG TPPLSPPFRP RCRHRPFGTN ETHRFPHHRI
              330 340 350 360
                  | | | |
SVNIIHRPPP HGHHPHGPPP HGHHPHGPPP HGHPPHGPPP
              370 380 390 400
                  | | | |
RHPPHGPPPH GHPPHGPPPH GHPPHGPPPH GHPPHGPPPH
              410 420 430 440
                  | | | |
GHPPHGHGFH DHGPCDPPSH KEGPQDLHQH AMGPPPKHPG
              450 460 470 480
                  | | | |
KRGPGKGHFP FHWRRIGSVY QLPPLQKGEV LPLPEANFPQ
              490 500 510 520
                  | | | |
LLLRNHTHPL KPEIQPFPQV ASERCPEEFN GEFAQLSKFF
      526
          |
PSTFPK
```

italic: signal sequence

double underscore: Pro-rich domain

single underscore: His-Pro (H/P) rich domain

The rabbit protein is encoded by a DNA molecule
having the sequence:

| | |
|---|---|
| | SEQ ID NO:4 |
| 1 | gcgccacact gcagtgttcg tgggctttga ctcccactga |
| | ctgcaaaact accaagccct |
| 61 | tggctgagaa agctctagac ctgatcaata aatggcgacg |
| | ggatggctac cttttccagt |
| 121 | tgctgcgagt cgctgatgcc cacttggacg gagcggaatc |
| | tgccactgtc tactatttag |
| 181 | tcttagatgt gaaagagact gactgttcag tgctatccag |
| | gaaacactgg gaagactgtg |
| 241 | acccagatct tactaaacgt ccatctcttg acgtgattgg |
| | gcaatgtaag gtgatagcta |
| 301 | ccagatattc ggatgaatat cagactctaa gattgaatga |
| | ctttaactgc accacgagtt |
| 361 | ccgtctcttc agccctggcc aacactaaag acagtcctgt |
| | tctctttgat ttcatcgagg |
| 421 | acacggagcc cttcagaaaa tccgcggaca aagccctgga |
| | ggtgtacaaa agtgaaagcg |
| 481 | aggcgtatgc ctctttcaga gtggaccggg tagagagagt |
| | cacaagggtg aaaggaggag |
| 541 | agagaaccaa ttactatgtg gacttctccg tgaggaactg |
| | ctccaggtct cacttccaca |
| 601 | gacaccccgc ctttgggtto tgcagagcag atctgtcctt |
| | tgatgtagaa gcctcgaact |
| 661 | tggaaaaccc agaagacgtt attataagct gtgaagtctt |
| | taactttgag gaacatggaa |
| 721 | acatcagtgg ttttcgaccc catttgggca agactccact |
| | tgggactgat ggatccagag |
| 781 | atcatcatca tccccacaag ccacataagt ttggatgccc |
| | acctccccaa gaaggggaag |
| 841 | atttctcgga aggaccacca cttcaaggtg gaaccccccc |
| | actctccccc cccttcaggc |
| 901 | caagatgtcg tcatcgccct tttggcacca atgaaaccca |
| | tcggttccct catcatcgaa |
| 961 | tttcagtgaa catcatccat aggccccctc cccatggaca |
| | tcacccccat gggccccctc |
| 1021 | cccatggaca tcacccccat gggccccctc cccatggaca |
| | tcctcctcat ggaccccctc |
| 1081 | cccgacatcc tccccatggg cctcctcccc atggacatcc |
| | cccccatgga ccccctcccc |
| 1141 | atggacatcc tcctcatgga ccccctcccc atggacatcc |
| | tccccatggg ccccctcccc |
| 1201 | atggacatcc tccccatggc catggtttcc atgaccatgg |
| | accctgtgac ccaccatccc |
| 1261 | ataaagaagg tccccaagac ctccatcagc atgccatggg |
| | accaccacct aagcacccag |
| 1321 | gaaagagagg tccaggtaaa ggacactttc ccttccactg |
| | gagaagaatt gggtctgttt |
| 1381 | accaactgcc cccactgcag aaaggtgaag tccttcccct |
| | tcccgaagcc aattttcccc |
| 1441 | agcttctctt gcggaaccac acccaccctc taaagcccga |
| | gatccagccc ttccctcagg |
| 1501 | tagcctctga gcgctgtcca gaggagttca atggtgagtt |
| | tgcacaactc tccaagtttt |
| 1561 | tcccatctac atttccaaaa tgaaatctga tttccttgat |
| | gggnaacaat gaatgatatt |
| 1621 | ctgtattagc accataaata aaatgtggcc atgatgaatg |
| | ca |

Preferred polypeptides are the H/P domain of human HPRG,

Preferred polypeptides are the H/P domain of human
HPRG,
(SEQ ID NO:5)
HPHKHHSHEQ HPHGHHPHAH HPHEHDTHRQ HPHGHHPHGH
HPHGHHPHGH HPHGHHPHCH DFQDYGPCDP PPHNQGHCCH
GHGPPPGHLR RRGPGKGPRP FHCRQIGSVY RLPPLRKGEV
LPLPEANFPS FPLPHHKHPL KPDNQPFP
and the H/P domain of rabbit HPRG,
(SEQ ID NO:6)
SVNIIHRPPP HGHHPHGPPP HGHHPHGPPP HGHPPHGPPP
RHPPHGPPPH GHPPHGPPPH GHPPHGPPPH GHPPHGPPPH
GHPPHGHGFH DHGPCDPPSHK

Further, homologues of the HPRG protein or of its domains (e.g., Borza et al., 1996. supra) or peptides thereof that share sequence similarity with HPRG also exhibit anti-angiogenic and anti-tumor activity.

Examples of such homologues are Plasmodium falciparum erythrocyte membrane protein-1, Plasmodium falciparum histidine-rich protein 2 (PfHRP2) and the histatin family of proteins.

A functional homologue must possess the biochemical and biological activity, preferably anti-angiogenic and anti-tumor activity which can be tested using in vitro or in vivo methods described herein. In view of this functional characterization, use of homologous HPRG proteins from other species, including proteins not yet discovered, falls within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. For example, preferred alignment would be with human HPRG protein H/P domain (SEQ ID NO:5) or rabbit HPRG protein H/P domain (SEQ ID NO:6), at least 30%, preferably at least 40%, more preferably at least 50%, even more preferably at least 60% and even more preferably at least 70, 80 or 90% of the amino acid residues are aligned. The amino acid residues (or nucleotides from the coding sequence) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to human or murine HPRG nucleic acid molecules. BLAST protein searches can be performed with the XBLAST program, score=50, wordlength=3 to obtain amino acid sequences homologous to HPRG protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.k

Thus, a homologue of the HPRG described above is characterized as having (a) functional activity of native HPRG, and (b) sequence similarity to a native HPRG when determined above, of at least about 30% (at the amino acid level), preferably at least about 50%, more preferably at least about 70%, even more preferably at least about 90%.

It is within the skill in the art to obtain and express such a protein using DNA probes based on the disclosed sequences of HPRG. Then, the protein's biochemical and biological activity can be tested readily using art-recognized methods such as those described herein. A biological assay of endothelial cell proliferation will indicate whether the homologue has the requisite activity to qualify as a “functional” homologue.

Peptide Compositions

A preferred composition is, or comprises, a biologically active peptide of HPRG characterized in that it possesses the binding and/or biological activity of HPRG. Such binding is to a ligand that is preferably a member of the following ligand classes:

(1) ligands belonging to the coagulation/fibrinolysis systems such as heparin, plasminogen, fibrinogen, vitronectin and thrombospondin. HPRG may bind similarly to other molecules that interact with these ligands. Thus, the present invention preferably includes novel any molecule that binds to the above-mentioned ligands.

(2) small ligands, such as heme or transition metal ions (zinc, copper and nickel), or

(3) cells such as T cells, macrophages and platelets.

Moreover, a biologically active peptide has HPRG activity in an in vitro or in vivo assay of binding or of biological activity such as those characterized herein. Preferably the peptide inhibits endothelial cell proliferation or migration, EC tube formation, angiogenesis or tumor growth at a level at least about 20% of the activity of full length HPRG.

A preferred peptide comprises a minimal consensus sequence [H/P][H/P]PHG (SEQ ID NO:7) that is derived from the comparison of the amino acid sequence of one or more domains of HPRG among different species. An addition variant of such a consensus sequence peptide has between 1-4 additional amino acids selected from H, P and G in any combination. Longer peptide multimers of the invention are described below.

The peptide may be capped at its N and C termini with an acyl (abbreviated “Ac”)and an amido (abbreviated “Am”) group, respectively, for example acetyl (CH₃CO—) at the N terminus and amido (—NH₂) at the C terminus.

A broad range of N-terminal capping functions, preferably in a linkage to the terminal amino group, is contemplated, for example:

formyl;

alkanoyl, having from 1 to 10 carbon atoms, such as acetyl, propionyl, butyryl;

alkenoyl, having from 1 to 10 carbon atoms, such as hex-3-enoyl;

alkynoyl, having from 1 to 10 carbon atoms, such as hex-5-ynoyl;

aroyl, such as benzoyl or 1-naphthoyl;

heteroaroyl, such as 3-pyrroyl or 4-quinoloyl;

alkylsulfonyl, such as methanesulfonyl;

arylsulfonyl, such as benzenesulfonyl or sulfanilyl;

heteroarylsulfonyl, such as pyridine-4-sulfonyl;

substituted alkanoyl, having from 1 to 10 carbon atoms, such as 4-aminobutyryl;

substituted alkenoyl, having from 1 to 10 carbon atoms, such as 6-hydroxy-hex-3-enoyl;

substituted alkynoyl, having from 1 to 10 carbon atoms, such as 3-hydroxy-hex-5-ynoyl;

substituted aroyl, such as 4-chlorobenzoyl or 8-hydroxy-naphth-2-oyl;

substituted heteroaroyl, such as 2,4-dioxo-1,2,3,4-tetrahydro-3-methyl-quinazolin-6-oyl;

substituted alkylsulfonyl, such as 2-aminoethanesulfonyl;

substituted arylsulfonyl, such as 5-dimethylamino-1-naphthalenesulfonyl;

substituted heteroarylsulfonyl, such as 1-methoxy-6-isoquinolinesulfonyl;

carbamoyl or thiocarbamoyl;

substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl;

substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

The C-terminal capping function can either be in an amide or ester bond with the terminal carboxyl. Capping functions that provide for an amide bond are designated as NR¹R² wherein R¹ and R² may be independently drawn from the following group:

hydrogen;

alkyl, preferably having from 1 to 10 carbon atoms, such as methyl, ethyl, isopropyl;

alkenyl, preferably having from 1 to 10 carbon atoms, such as prop-2-enyl;

alkynyl, preferably having from 1 to 10 carbon atoms, such as prop-2-ynyl;

substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;

substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;

substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;

aroylalkyl having up to 10 carbon atoms, such as phenacyl or 2-benzoylethyl;

aryl, such as phenyl or 1-naphthyl;

heteroaryl, such as 4-quinolyl;

alkanoyl having from 1 to 10 carbon atoms, such as acetyl or butyryl;

aroyl, such as benzoyl;

heteroaroyl, such as 3-quinoloyl;

OR′ or NR′R″ where R′ and R″ are independently hydrogen, alkyl, aryl, heteroaryl, acyl, aroyl, sulfonyl, sulfinyl, or SO₂—R′″ or SO—R′″ where R′″ is substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

Either the N-terminal or the C-terminal capping function, or both, may be of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules.

Production of Peptides and Derivatives

General Chemical Synthetic Procedures

The peptides of the invention may be prepared using recombinant DNA technology. However, given their length, they are preferably prepared using solid-phase synthesis, such as that generally described by Merrifield, J. Amer. Chem. Soc., 85:2149-54 (1963), although other equivalent chemical syntheses known in the art are also useful. Solid-phase peptide synthesis may be initiated from the C-terminus of the peptide by coupling a protected α-amino acid to a suitable resin. Such a starting material can be prepared by attaching an α-amino-protected amino acid by an ester linkage to a chloromethylated resin or to a hydroxymethyl resin, or by an amide bond to a BHA resin or MBHA resin.

Such methods, well-known in the art, are disclosed, for example, in U.S. Pat. No. 5,994,309 (issued Nov. 30, 1999) which is incorporated by reference in its entirety.

Amino Acid Substitution and Addition Variants

Also included in this invention are peptides in which at least one amino acid residue and preferably, only one, has been removed and a different residue inserted in its place compared to the native sequence. For a detailed description of protein chemistry and structure, see Schulz, G. E. et al., Principles of Protein Structure, Springer-Verlag, New York, 1979, and Creighton, T. E., Proteins: Structure and Molecular Principles, W. H. Freeman & Co., San Francisco, 1984, which are hereby incorporated by reference. The types of substitutions which may be made in the peptide molecule of the present invention are conservative substitutions and are defined herein as exchanges within one of the following groups:

1. Small aliphatic, nonpolar or slightly polar residues: e.g., Ala, Ser, Thr, Gly;

2. Polar, negatively charged residues and their amides: e.g., Asp, Asn, Glu, Gln;

3. Polar, positively charged residues: e.g., His, Arg, Lys;

Pro, because of its unusual geometry, tightly constrains the chain. Substantial changes in functional properties are made by selecting substitutions that are less conservative, such as between, rather than within, the above groups (or two other amino acid groups not shown above), which will differ more significantly in their effect on maintaining (a) the structure of the peptide backbone in the area of the substitution (b) the charge or hydrophobicity of the molecule at the target site, or (c) the bulk of the side chain. Most substitutions according to the present invention are those that do not produce radical changes in the characteristics of the peptide molecule. Even when it is difficult to predict the exact effect of a substitution in advance of doing so, one skilled in the art will appreciate that the effect can be evaluated by routine screening assays, preferably the biological assays described below. Modifications of peptide properties including redox or thermal stability, hydrophobicity, susceptibility to proteolytic degradation or the tendency to aggregate with carriers or into multimers are assayed by methods well known to the ordinarily skilled artisan.

The present invention provides methods to inhibit or reduce angiogenesis, tumor growth, EC proliferation, EC migration or EC tube formation.

The invention also provides pharmaceutical compositions comprising fragments, peptides, conformers, antibodies, biological equivalents or derivatives of HPRG.

The HPRG used in the present invention can be derived from any organism that produces it in nature such as rabbits or, preferably, humans. The nucleotide sequence (SEQ ID NO:2 and amino acid sequence (SEQ ID NO: 1) of human HPRG are available from GenBank (GenBank Accession number M1349, and Swiss Prot number: PO₄₁₉₆).

HPRG is isolated from a body fluid such as blood and urine, though it can also be obtained from other sources such as tissue extracts of as a product of a cell line growing in culture that produces “native” HPRG or that has been genetically modified with DNA encoding native HPRG or a functional derivative thereof to express this protein or a functional derivative thereof such as a domain or shorter fragment.

HPRG, fragments or derivatives are chemically synthesized, or produced by recombinant methods. Recombinant techniques known in the art include, but are not limited to DNA amplification using PCR of a cDNA library for example by reverse transcription of mRNA in cells extracts followed by PCR.

Basic texts disclosing general methods of molecular biology, all of which are incorporated by reference, include: Sambrook, J. et al., Molecular Cloning: A Laboratory Manual, ₂^{nd} Edition, Cold Spring Harbor Press, Cold Spring Harbor, N.Y., 1989; Ausubel, F. M. et al. Current Protocols in Molecular Biology, Vol. 2, Wiley-Interscience, New York, (current edition); Kriegler, Gene Transfer and Expression: A Laboratory Manual (1990); Glover, D. M., ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J. D. et al., Recombinant DNA, ₂^{nd} Ed., Scientific American Books, New York, 1992; and Old, RW et al., Principles of Gene Manipulation. An Introduction to Genetic Engineering, 2^{nd} Ed., University of California Press, Berkeley, Calif. (1981).

Fragments of HPRG are be obtained by controlled protease reaction (Borza D-B. et al., Biochemistry, 1996, 35; 1925-1934). An example of such is limited plasmin digestion of HPRG followed by partial reduction with dithiothreitol to create fragments of HPRG that inhibit angiogenesis, EC proliferation, migration or tube formation and/or tumor growth.

Chemical Derivatives of HPRG

“Chemical derivatives” of HPRG contain additional chemical moieties not normally a part of the protein. Covalent modifications of the polypeptide are included within the scope of this invention. Such derivatized moieties may improve the solubility, absorption, biological half life, and the like. Moieties capable of mediating such effects are disclosed, for example, in Remington 's Pharmaceutical Sciences, 16^{th} ed., Mack Publishing Co., Easton, Pa. (1980).

Such modifications may be introduced into the molecule by reacting targeted amino acid residues of the polypeptide with an organic derivatizing agent that is capable of reacting with selected side chains or terminal residues. Another modification is cyclization of the protein.

Cysteinyl residues most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate (pH 5.5-7.0) which agent is relatively specific for the histidyl side chain. p-bromophenacyl bromide also is useful; the reaction is preferably performed in 0.1 M sodium cacodylate at pH 6.0.

Lysinyl and amino terminal residues are derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKₐ of the guanidine functional group. Furthermore, these reagents may react with the groups of lysine as well as the arginine ε-amino group.

Modification of tyrosyl residues has permits introduction of spectral labels into a peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively.

Carboxyl side groups, aspartyl or glutamyl, may be selectively modified by reaction with carbodiimides (R—N═C═N—R′) such as 1-cyclohexyl-3-(2-morpholinyl-(4-ethyl) carbodiimide or 1-cthyl-3-(4-azonia-4,4-dimethylpentyl) carbodiimide. Furthermore, aspartyl and glutamyl residues can be converted to asparaginyl and glutaminyl residues by reaction with ammonia.

Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention.

Derivatization with bifunctional agents is useful for cross-linking the peptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of the hydroxyl groups of seryl or threonyl residues, methylation of the α-amino groups of lysine, arginine, and histidine side chains (T. E. Creighton, Proteins: Structure and Molecule Properties, W. H. Freeman & Co., San Francisco, pp. 79-86 (1983)), acetylation of the N-terminal amine, and, in some instances, amidation of the C-terminal carboxyl groups.

Also included are peptides wherein one or more D-amino acids are substituted for one or more L-amino acids.

Multimeric Peptides

The present invention also includes longer peptides built from repeating units of one or more sequences from the H/P domain of the HPRG protein that have anti-angiogenic activity. The preferred peptide unit of such a multimer is a pentapeptide, preferably His-His-Pro-His-Gly (SEQ ID NO:8), His-Pro-Pro-His-Gly (SEQ ID NO:9), or Pro-Pro-Pro-His-Gly (SEQ ID NO:10).

Addition variants of these peptide units preferably include from 1-4 amino acids selected from His, Pro and Gly.

Such multimers may be built from any of the peptides or their variants described herein. Moreover, a peptide multimer may comprise different combinations of peptide monomers (either from the native sequence of human or rabbit HPRG or addition variants thereof. Such oligomeric or multimeric peptides can be made by chemical synthesis or by recombinant DNA techniques as discussed herein. When produced by chemical synthesis, the oligomers preferably have from 2-12 repeats, more preferably 2-8 repeats of the core peptide sequence, and the total number of amino acids in the multimer should not exceed about 110 residues (or their equivalents, when including linkers or spacers).

A preferred synthetic chemical peptide multimer has the formula

P¹ₙ

wherein P¹ is a pentapeptides corresponding to five sequential amino acids from the H/P domain of a mammalian HPRG protein, or substitution or addition variants of these pentapeptides, wherein n=2-8, and wherein the pentapeptide alone or in multimeric form has the biological activity of inhibiting cell invasion, endothelial tube formation or angiogenesis in an in vitro or in vivo bioassay of such activity.

In another embodiment, a preferred synthetic chemical peptide multimer has the formula

(P¹-Xₘ)ₙ-P²

P¹ and P² are pentapeptides corresponding to five sequential amino acids from the H/P domain of a mammalian HPRG protein, or addition variants of these pentapeptides, wherein (a) P¹ and P² may be the same or different; moreover, each occurrence of P¹ in the multimer may be different pentapeptides (or variant);

(b) X is C₁-C₅ alkyl, C₁-C₅ alkenyl, C₁-C₅ alkynyl, C₁-C₅ polyether containing up to 4 oxygen atoms, wherein m=0 or 1 and n=1-7; X may also be Gly_{z} wherein, z=1-6, and wherein the pentapeptide alone or in multimeric form has the biological activity of inhibiting cell invasion, endothelial tube formation or angiogenesis in an in vitro or in vivo bioassay of such activity.

When produced recombinantly, spacers are Gly_{z} as described above, where z=1-6, and the multimers may have as many repeats of the core peptide sequence as the expression system permits, for example from two to about 100 repeats. A preferred recombinantly produced peptide multimer has the formula:

(P′-Gly_{z})ₙ-P²

wherein:

(a) P¹ and P² are pentapeptides corresponding to five sequential amino acids from the H/P domain of a mammalian HPRG protein, or addition variants of these pentapeptides, wherein P¹ and P² may be the same or different; moreover, each occurrence of P¹ in the multimer may be different pentapeptides (or variant);

wherein n=1-100 and z=0-6;

and wherein the pentapeptide alone or in multimeric form has the biological activity of inhibiting cell invasion, endothelial tube formation or angiogenesis in an in vitro or in vivo bioassay of such activity.

In the foregoing peptide multimers, P¹ and P² is preferably SEQ ID NO:8, 9 or 10.

The multimer is optionally capped at its N- and C-termini,

It is understood that such multimers may be built from any of the peptides or variants described herein. Although it is preferred that the addition variant monomeric units of the multimer have the biological activity described above, that is not necessary as long as the multimer to which they contribute has the activity.

Diagnostic and Prognostic Compositions

The peptides of the invention can be detectably labeled and used, for example, to detect a peptide binding protein ligand or a cellular binding site/receptor (such as the binding sites on T cells, macrophages or platelets as described above, whether on the surface or in the interior of a cell. The fate of the peptide during and after binding can be followed in vitro or in vivo by using the appropriate method to detect the label. The labeled peptide may be utilized in vivo for diagnosis and prognosis, for example to image occult metastatic foci or for other types of in situ evaluations.

The term “diagnostically labeled” means that the polypeptide or peptide has attached to it a diagnostically detectable label. There are many different labels and methods of labeling known to those of ordinary skill in the art, described below. General classes of labels which can be used in the present invention include radioactive isotopes, paramagnetic isotopes, and compounds which can be imaged by positron emission tomography (PET), fluorescent or colored compounds, etc. Suitable detectable labels include radioactive, fluorescent, fluorogenic, chromogenic, or other chemical labels. Useful radiolabels (radionuclides), which are detected simply by gamma counter, scintillation counter or autoradiography include ³H, ¹²⁵I, ¹³¹I, ³⁵S and ¹⁴C. ¹³¹I is also a useful therapeutic isotope (see below).

A number of U.S. patents, incorporated by reference herein, disclose methods and compositions for complexing metals to larger molecules, including description of useful chelating agents. The metals are preferably detectable metal atoms, including radionuclides, and are complexed to proteins and other molecules. These documents include: U.S. Pat. No. 5,627,286 (Heteroatom-bearing ligands and metal complexes thereof); U.S. Pat. No. 5,618,513 (Method for preparing radiolabeled peptides); U.S. Pat. No. 5,567,408; U.S. Pat. No. 5,443,816 (Peptide-metal ion pharmaceutical preparation and method); U.S. Pat. No. 5,561,220 (Tc-^{99m} labeled peptides for imaging inflammation).

Common fluorescent labels include fluorescein, rhodamine, dansyl, phycoerythrin, phycocyanin, allophycocyanin, o-phthaldehyde and fluorescamine. The fluorophore, such as the dansyl group, must be excited by light of a particular wavelength to fluoresce. See, for example, Haugland, Handbook of Fluorescent Probes and Research Chemicals, Sixth Ed., Molecular Probes, Eugene, Oreg., 1996). Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Similarly, fluorophores may also be coupled to thiols using maleimide, iodoacetamide, and aziridine-reactive groups. The long wavelength rhodamines, which are basically Rhodamine Green™ derivatives with substituents on the nitrogens, are among the most photostable fluorescent labeling reagents known. Their spectra are not affected by changes in pH between 4 and 10, an important advantage over the fluoresceins for many biological applications. This group includes the tetramethylrhodamines, X-rhodamines and Texas Red™ derivatives. Other preferred fluorophores for derivatizing the peptide according to this invention are those which are excited by ultraviolet light. Examples include cascade blue, coumarin derivatives, naphthalenes (of which dansyl chloride is a member), pyrenes and pyridyloxazole derivatives. Also included as labels are two related inorganic materials that have recently been described: semiconductor nanocrystals, comprising, for example, cadmium sulfate (Bruchez, M. et al., Science 281:2013-2016 (1998), and quantum dots, e.g., zinc-sulfide-capped Cd selenide (Chan, W. C. W. et al., Science 281:2016-2018 (1998)).

In yet another approach, the amino group of the peptide is allowed to react with reagents that yield fluorescent products, for example, fluorescamine, dialdehydes such as o-phthaldialdehyde, naphthalene-2,3-dicarboxylate and anthracene-2,3-dicarboxylate. 7-nitrobenz-2-oxa-1,3-diazole (NBD) derivatives, both chloride and fluoride, are useful to modify amines to yield fluorescent products.

The peptides of the invention can also be labeled for detection using fluorescence-emitting metals such as ¹⁵²Eu, or others of the lanthamide series. These metals can be attached to the peptide using such metal chelating groups as diethylenetriaminepentaacetic acid (DTPA, see Example X, infra) or ethylenediaminetetraacetic acid (EDTA). DTPA, for example, is available as the anhydride, which can readily modify the NH₂-containing peptides of this invention.

For in vivo diagnosis or therapy, radionuclides may be bound to the peptide either directly or indirectly using a chelating agent such as DTPA and EDTA. Examples of such radionuclides are ⁹⁹Tc, ¹²³I, ¹²⁵I, ¹³¹I, ¹¹¹In, ⁹⁷Ru, ⁶⁷Cu, ⁶⁷Ga, ⁶⁸Ga, ⁷²As, ⁸⁹Zr, ⁹⁰Y and ²⁰¹Tl. Generally, the amount of labeled peptide needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg.

The peptide can also be made detectable by coupling to a phosphorescent or a chemiluminescent compound. The presence of the chemiluminescent-tagged peptide is then determined by detecting the presence of luminescence that arises during the course of a chemical reaction. Examples of particularly useful chemiluminescers are luminol, isoluminol, theromatic acridinium ester, imidazole, acridinium salt and oxalate ester. Likewise, a bioluminescent compound may be used to label the peptides. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In yet another embodiment, calorimetric detection is used, based on chromogenic compounds which have, or result in, chromophores with high extinction coefficients.

In situ detection of the labeled peptide may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

For diagnostic in vivo radioimaging, the type of detection instrument available is a major factor in selecting a radionuclide. The radionuclide chosen must have a type of decay which is detectable by a particular instrument. In general, any conventional method for visualizing diagnostic imaging can be utilized in accordance with this invention. Another factor in selecting a radionuclide for in vivo diagnosis is that its half-life be long enough so that the label is still detectable at the time of maximum uptake by the target tissue, but short enough so that deleterious irradiation of the host is minimized. In one preferred embodiment, a radionuclide used for in vivo imaging does not emit particles, but produces a large number of photons in a 140-200 keV range, which may be readily detected by conventional gamma cameras.

In vivo imaging may be used to detect occult metastases which are not observable by other methods. Imaging could be used to stage tumors non-invasively or to detect other diseases which are associated with the presence of increased levels of a HPRG-binding site or ligand.

Peptidomimetics

A preferred type of chemical derivative of the peptides described herein is a peptidomimetic compound which mimics the biological effects of HPRG or of a biologically active peptide thereof. A peptidomimetic agent may be an unnatural peptide or a non-peptide agent that recreates the stereospatial properties of the binding elements of HPRG such that it has the binding activity or biological activity of HPRG. Similar to biologically active HPRG peptides, a peptidomimetic will have a binding face (which interacts with any ligand to which HPRG binds) and a non-binding face.

Again, similar to HPRG or its peptide, the non-binding face of a peptidomimetic will contain functional groups which can be modified by various therapeutic and diagnostic moieties without modifying the binding face of the peptidomimetic (again, I do not see the description of this for the protein and peptide)???. A preferred embodiment of a peptidomimetic would contain an aniline on the non-binding face of the molecule. The NH₂-group of an aniline has a pKa˜4.5 and could therefore be modified by any NH₂-selective reagent without modifying any NH₂ functional groups on the binding face of the peptidomimetic. Other peptidomimetics may not have any NH₂ functional groups on their binding face and therefore, any NH₂, without regard for pKₐ could be displayed on the non-binding face as a site for conjugation. In addition other modifiable functional groups, such as —SH and —COOH could be incorporated into the non-binding face of a peptidomimetic as a site of conjugation. A therapeutic or diagnostic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptide nature.

Peptidomimetic compounds, either agonists, substrates or inhibitors, have been described for a number of bioactive peptides such as opioid peptides, VIP, thrombin, HIV protease, etc. Methods for designing and preparing peptidomimetic compounds are known in the art (Hruby, V. J., Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that possess at least the binding capacity and specificity of the HPRG peptides and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the cystallographically-derived three-dimensional structure of a peptide of the invention either free or bound in complex with a ligand such as (a) heparin, plasminogen, fibrinogen, vitronectin and thrombospondin or (b) small ligands, such as heme and transition metal ions (zinc, copper and nickel). Alternatively, the structure of a peptide of the invention bound to its ligand can be gained by the techniques of nuclear magnetic resonance spectroscopy. The better knowledge of the stereochemistry of the interaction of the peptide with its ligand or receptor will permit the rational design of such peptidomimetic agents. The structure of a peptide or protein of the invention in the absence of ligand could also provide a scaffold for the design of mimetic molecules.

Antibodies Specific for Epitopes of HPRG

The present invention provides antibodies, both polyclonal and monoclonal, reactive with an epitope of HPRG, preferably, an epitope of the H/P domain. The antibodies, referred to herein as “anti-H/P antibodies” may be xenogeneic, allogeneic, syngeneic, or modified forms thereof, such as humanized or chimeric antibodies. Antiidiotypic antibodies specific for the idiotype of an anti-HPRG antibody are also included.

In the following description, reference will be made to various methodologies known to those of skill in the art of immunology. Publications and other materials setting forth such known methodologies to which reference is made are incorporated herein by reference in their entireties as though set forth in full. Standard reference works setting forth the general principles of immunology include A. K. Abbas et al., Cellular and Molecular Immunology (Fourth Ed.), W. B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C. V. Mosby Co., St. Louis, Mo. (1999); Klein, J., Imnmunology, Blackwell Scientific Publications, Inc., Cambridge, Mass., (1990).

Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbiol., Immunol. Volume 119, 1985; Bona, C. et al., CRC Crit. Rev. Immunol., pp. 33-81 (1981); Jerne, N K, Ann. Immunol. 125C:373-389 (1974); Jerne, NK, In: Idiotypes—Antigens on the Inside, Westen-Schnurr, I., ed., Editiones Roche, Basel, 1982, Urbain, J et al., Ann. Immunol. 133D:179-(1982); Rajewsky, K. et al., Ann. Rev. Immunol. 1:569-607 (1983)

The term “antibody” is also meant to include both intact molecules as well as fragments thereof that include the antigen-binding site and are capable of binding to a HPRG epitope. These include, Fab and F(ab′)₂ fragments which lack the Fc fragment of an intact antibody, clear more rapidly from the circulation, and may have less non-specific tissue binding than an intact antibody (Wahl et al., J. Nucl. Med. 24:316-325 (1983)). Also included are Fv fragments (Hochman, J. et al. (1973) Biochemistry 12:1130-1135; Sharon, J. et al.(1976) Biochemistry 15:1591-1594).). These various fragments are to be produced using conventional techniques such as protease cleavage or chemical cleavage (see, e.g., Rousseaux et al., Meth. Enzymol., 121:663-69 (1986))

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography (see Zola et al., supra).

The immunogen used to produce the present anti-H/P antibodies may comprise the complete HPRG protein, or fragments or derivatives thereof. Preferred immunogens comprise all or a part of the H/P central domain of HPRG. Immunogens comprising this domain are produced in a variety of ways known in the art, e.g., expression of cloned genes using conventional recombinant methods, isolation from cells of origin, cell populations expressing high levels of HPRG, etc.

The mAbs may be produced using conventional hybridoma technology, such as the procedures introduced by Kohler and Milstein (supra) and modifications thereof (see above references). An animal, preferably a mouse is primed by immunization with an immunogen as above to elicit the desired antibody response in the primed animal.

B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g., by immunoassay techniques using the HPRG protein Positive clones are subcloned, e.g., by limiting dilution, and the mAbs are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single mAb can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost CR et al,. J. Biol Chem. 1994 269:26267-26273; U.S. Pat. No. 4,704,692, 4,853,871, 4,94,6778, 5,260,203, 5,455,030). DNA sequences encoding the V regions of the H chain and the L chain are ligated to a linker encoding at least about 4 amino acids (typically small neutral amino acids). The protein encoded by this fusion allows assembly of a functional variable region that retains the specificity and affinity of the original antibody.

For in vivo use, particularly for injection into humans, it is desirable to decrease the immunogenicity of the mAb by humanizing the antibodies using methods known in the art. The humanized antibody may be the product of an animal having transgenic human Ig Constant region genes (see for example WO 90/10077 and WO 90/04036). Alternatively, the antibody of interest may be genetically engineered to substitute the CH₁, CH₂, CH₃, hinge domains, and/or the framework domain with the corresponding human sequence (see WO 92/02190).

Antibodies can be selected for particular desired properties. In the case of an antibody to be used for therapy, antibody screening procedures can include any of the in vitro or in vivo bioassays that measure angiogenesis, cell invasion, and the like. Moreover, the antibodies may be screened in various of the tumor models described herein to see if they promote or inhibit angiogenesis (or resultant tumor growth or metastasis). In this way, antibodies that are HPRG mimics or antagonists can be selected. Thus, the present invention includes therapeutic antibodies (discussed in more detail below) that promote angiogenesis by binding to and otherwise inhibiting the action of HPRG or its H/P domain.

Use of Antibodies to Detect Free H/P Domain of HPRG

Antibodies specific for an epitope of the H/P domain are useful in immunoassays to detect molecules containing these epitopes in a body fluid or sample, preferably serum or plasma. Such antibodies would detect HPRG, a cleaved H/P domain of HPRG or an epitope-bearing fragment of the domain. Thus, if proteolysis in the tumor milieu results in release of the H/P domain plasma (just in case proteolysis releases free H/P in the tumor milieu) or in tissue.

By measuring the levels of H/P domain released from HPRG, the antibodies and immunoassays of this invention are used diagnostically to monitor the progress of a disease, where H/P domain levels may reflect the amount of tumor tissue present.

Any conventional immunoassay known in the art may be employed for this purpose, though Enzyme Immunoassays such as ELISA are preferred. Immunoassay methods are also described in Coligan, J. E. et al., eds., Current Protocols in Immunology, Wiley-Interscience, New York 1991 (or current edition); Butt, W. R. (ed.) Practical Immunoassay. The State of the Art, Dekker, New York, 1984; Bizollon, Ch. A., ed., Monoclonal Antibodies and New Trends in Immulloassays, Elsevier, New York, 1984; Butler, J. E., ELISA (Chapter 29), In: van Oss, C. J. et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

In Vitro Testing of Compositions

A. Assay for Endothelial Cell Migration

For EC migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as human umbilical vein endothelial cells (HUVEC), which have been detached from monolayer culture using trypsin, are diluted to a final concentration of about 10⁶ cells/mL with serum-free media and 0.2 mL of this cell suspension is added to the upper chamber of each transwell. Inhibitors to be tested are added to both the upper and lower chambers, and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated.

B. Biological Assay of Anti-Invasive Activity

The compositions of the invention are tested for their anti-invasive capacity. The ability of cells such as ECs or tumor cells (e.g., PC-3 human prostatic carcinoma) cells to invade through a reconstituted basement membrane (Matrigel®) in an assay known as a Matrigel® invasion assay system as described in detail by Kleinman et al., Biochemistry 25: 312-318,1986 and Parish et al., Int. J. Cancer 52:378-383,1992. Matrigel® is a reconstituted basement membrane containing type IV collagen, laminin, heparan sulfate proteoglycans such as perlecan, which bind to and localize bFGF, vitronectin as well as transforming growth factor-β (TGFβ), urokinase-type plasminogen activator (uPA), tissue plasminogen activator (tPA), and the serpin known as plasminogen activator inhibitor type 1 (PAI-1) (Chambers et al., Canc. Res. 55:1578-1585, 1995). It is accepted in the art that results obtained in this assay for compounds which target extracellular receptors or enzymes are predictive of the efficacy of these compounds in vivo (Rabbani et al., Int. J. Cancer 63: 840-845, 1995).

Such assays employ transwell tissue culture inserts. Invasive cells are defined as cells which are able to traverse through the Matrigel® and upper aspect of a polycarbonate membrane and adhere to the bottom of the membrane. Transwells (Costar) containing polycarbonate membranes (8.0 μm pore size) are coated with Matrigel® (Collaborative Research), which has been diluted in sterile PBS to a final concentration of 75 μg/mL (60 μL of diluted Matrigel® per insert), and placed in the wells of a 24-well plate. The membranes are dried overnight in a biological safety cabinet, then rehydrated by adding 100 μL of DMEM containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/ antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DiffQuik® (Giemsa stain) and the upper chamber is then scraped using a cotton swab to remove the Matrigel® and any cells which did not invade through the membrane. The membranes are detached from the transwell using an X-acto blade, mounted on slides using Permount and cover-slips, then counted under a high-powered (400×) field. An average of the cells invaded is determined from 5-10 fields counted and plotted as a function of inhibitor concentration.

C. Tube-Formation Assays of Anti-Angiogenic Activity

The compounds of this invention are tested for their anti-angiogenic activity in one of two different assay systems in vitro.

Endothelial cells, for example, human umbilical vein endothelial cells (HUVEC) or human microvascular endothelial cells (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/ mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch).

This assay is recognized in the art to be predictive of angiogenic (or anti-angiogenic) efficacy in vivo (Min, H Y et al., Cancer Res. 56: 2428-2433,1996). In an alternate assay, endothelial cell tube formation is observed when endothelial cells are cultured on Matrigel® (Schnaper et al., J. Cell. Physiol. 165:107-118 1995). Endothelial cells (1×10⁴ cells/well) are transferred onto Matrigel®-coated 24-well plates, and tube formation is quantitated after 48 hrs. Inhibitors are tested by adding them either at the same time as the endothelial cells or at various time points thereafter. Tube formation can also be stimulated by adding (a) angiogenic growth factors such as bFGF or VEGF, (b) differentiation stimulating agents (e.g.,. PMA) or (c) a combination of these.

This assay models angiogenesis by presenting to the endothelial cells a particular type of basement membrane, namely the layer of matrix which migrating and differentiating endothelial cells might be expected to first encounter. In addition to bound growth factors, the matrix components found in Matrigel® (and in basement membranes in situ) or proteolytic products thereof may also be stimulatory for endothelial cell tube formation which makes this model complementary to the fibrin gel angiogenesis model previously described (Blood and Zetter, Biochim. Biophys. Acta 1032:89-118, 1990; Odedra and Weiss, Pharmac. Ther. 49:111-124, 1991). The compounds of this invention inhibit endothelial cell tube formation in both assays, which suggests that the compounds will also have anti-angiogenic activity.

D. Assays for the Inhibition of Proliferation

The ability of the compounds of the invention to inhibit the proliferation of EC's may be determined in a 96-well format. Type I collagen (gelatin) is used to coat the wells of the plate (0.1-1 mg/mL in PBS, 0.1 mL per well for 30 minutes at room temperature). After washing the plate (3× w/PBS), 3-6,000 cells are plated per well and allowed to attach for 4 hrs (37° C./5% CO₂) in Endothelial Growth Medium (EGM; Clonetics) or M199 media containing 0.1-2% FBS. The media and any unattached cells are removed at the end of 4 hrs and fresh media containing bFGF (1-10 ng/mL) or VEGF (1-10 ng/mL) is added to each well. Compounds to be tested are added last and the plate is allowed to incubate (37° C./5% CO₂) for 24-48 hrs. MTS (Promega) is added to each well and allowed to incubate from 1-4 hrs. The absorbance at 490 nm, which is proportional to the cell number, is then measured to determine the differences in proliferation between control wells and those containing test compounds.

A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Compounds to be tested are included in some of the wells. After 24 hrs, MTS is added to the plate and the assay developed and read as described above.

E. Assays of Cytotoxicity

The anti-proliferative and cytotoxic effects of the compositions may be determined for various cell types including tumor cells, ECs, fibroblasts and macrophages. This is especially useful when testing a compound of the invention which has been conjugated to a therapeutic moiety such as a radiotherapeutic or a toxin. For example, a conjugate of one of the compositions with Bolton-Hunter reagent which has been iodinated with ¹³¹I, would be expected to inhibit the proliferation of cells expressing an HPRG binding site/receptor (most likely by inducing apoptosis). Anti-proliferative effects would be expected against tumor cells and stimulated endothelial cells but, under some circumstances not quiescent endothelial cells or normal human dermal fibroblasts. Any anti-proliferative or cytotoxic effects observed in the normal cells would represent non-specific toxicity of the conjugate.

A typical assay would involve plating cells at a density of 5-10,000 cells per well in a 96-well plate. The compound to be tested is added at a concentration 10× the IC₅₀ measured in a binding assay (this will vary depending on the conjugate) and allowed to incubate with the cells for 30 minutes. The cells are washed 3× with media, then fresh media containing [³H]thymidine (1 μCi/mL) is added to the cells and they are allowed to incubate at 37° C. in 5% CO₂ for 24 and 48 hours. Cells are lysed at the various time points using 1 M NaOH and counts per well determined using a β-counter. Proliferation may be measured non-radioactively using MTS reagent or CyQuant® to measure total cell number. For cytotoxicity assays (measuring cell lysis), a Promega 96-well cytotoxicity kit is used. If there is evidence of anti-proliferative activity, induction of apoptosis may be measured using TumorTACS (Genzyme).

Caspase-3 Activity

The ability of the compounds of the invention to promote apoptosis of EC's may be determined by measuring activation of caspase-3. Type I collagen (gelatin) is used to coat a P100 plate and 5×10⁵ ECs are seeded in EGM containing 10% FBS. After 24 hours (at 37° C. in 5% CO₂) the medium is replaced by EGM containing 2% FBS, 10 ng/ml bFGF and the desired test compound. The cells are harvested after 6 hours, cell lysates prepared in 1% Triton and assayed using the EnzChek® Caspase-3 Assay Kit #1 (Molecular Probes) according to the manufactures' instructions.

In vivo Study of the HPRG Peptides

A. Corneal Angiogenesis Model

The protocol used is essentially identical to that described by Volpert et al. (J. Clin. Invest. 98:671-679 (1996)). Briefly, female Fischer rats (120-140 gms) are anesthetized and pellets (5 μl) comprised of Hydron®, bFGF (150 nM), and the compounds to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

B. Matrigel® Plug Assay

This assay is performed essentially as described by Passaniti et al. (Lab Invest. 67:519-528 (1992). Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+ heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-endothelial cell adhesion molecule or PECAM”) may also be used to confirm neovessel formation and microvessel density in the plugs.

C. Chick Chorioallantoic Membrane (CAM) Angiogenesis Assay

This assay is performed essentially as described by Nguyen et al. (Microvascular Res. 47:31-40 (1994)). A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

D. In Vivo Assessment Angiogenesis inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel® and then injected into the flank of a mouse following the protocol described in Sec. B., above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenize with a tissue homogenizer.

E. Xenograft Model of Subcutaneous (s.c.) Tumor Growth

Nude mice are inoculated with MDA-MB-231 cells (human breast carcinoma) and Matrigel® (1×10⁶ cells in 0.2 mL) s.c. in the right flank of the animals. The tumors are staged to 200 mm³ and then treatment with a test composition is initiated (100 μg/animal/day given q.d. IP). Tumor volumes are obtained every other day and the animals are sacrificed after 2 weeks of treatment. The tumors are excised, weighed and paraffin embedded. Histological sections of the tumors are analyzed by H and E, anti-CD3 1, Ki-67, TUNEL, and CD68 staining.

F. Xenograft Model of Metastasis

The compounds of this invention are also tested for inhibition of late metastasis using an experimental metastasis model (Crowley, C. W. et al., Proc. Natl. Acad. Sci. USA 90 5021-5025 (1993)). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This approach permits utilization of either of these markers (fluorescence detection of GFP or histochemical calorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected iv into the tail veins of nude (nu/nu) mice. Animals are treated with a test composition at 100 μg/animal/day given q.d. IP. Single metastatic cells and foci are visualized and quantitated by fluorescence microscopy or light microscopic histochemistry or by grinding the tissue and quantitative colorimetric assay of the detectable label.

G. Inhibition of Spontaneous Metastasis In Vivo by HPRG and Functional Derivatives

The rat syngeneic breast cancer system (Xing et al., Int. J. Cancer 67:423-429 (1996) employs Mat BIII rat breast cancer cells. Tumor cells, for example about 10⁶ suspended in 0.1 mL PBS, are inoculated into the mammary fat pads of female Fisher rats. At the time of inoculation, a 14-day Alza osmotic mini-pump is implanted intraperitoneally to dispense the test compound. The compound is dissolved in PBS (e.g., 200 mM stock), sterile filtered and placed in the minipump to achieve a release rate of about 4 mg/kg/day. Control animals receive vehicle (PBS) alone or a vehicle control peptide in the minipump. Animals are sacrificed at about day 14.

Therapeutic Outcomes

In the rats treated with the active compounds of the present invention, significant reductions in the size of the primary tumor and in the number of metastases in the spleen, lungs, liver, kidney and lymph nodes (enumerated as discrete foci) are observed. Histological and immunohistochemical analysis reveal increased necrosis and signs of apoptosis in tumors in treated animals. Large necrotic areas are seen in tumor regions lacking neovascularization. Human or rabbit HPRG and their derivatives to which ¹³¹I is conjugated (either 1 or 2 I atoms per molecule of peptide) are effective radiotherapeutics and are found to be at least two-fold more potent than the unconjugated polypeptides. In contrast, treatment with control peptides fails to cause a significant change in tumor size or metastasis.

H. 3LL Lewis Lung Carcinoma: Primary Tumor Growth

This tumor line arose spontaneously in 1951 as carcinoma of the lung in a C57BL/6 mouse (Cancer Res 15:39, 1955. See, also Malave, I. et al., J. Nat'l. Canc. Inst. 62:83-88 (1979)). It is propagated by passage in C57BL/6 mice by subcutaneous (sc) inoculation and is tested in semiallogeneic C57BL/6× DBA/2 F₁ mice or in allogeneic C3H mice. Typically six animals per group for subcutaneously (sc) implant, or ten for intramuscular (im) implant are used. Tumor may be implanted sc as a 2-4 mm fragment, or im or sc as an inoculum of suspended cells of about 0.5-2×10⁶-cells. Treatment begins 24 hours after implant or is delayed until a tumor of specified size (usually approximately 400 mg) can be palpated. The test compound is administered ip daily for 11 days

Animals are followed by weighing, palpation, and measurement of tumor size. Typical tumor weight in untreated control recipients on day 12 after iminoculation is 500-2500 mg. Typical median survival time is 18-28 days. A positive control compound, for example cyclophosphamide at 20 mg/kg/injection per day on days 1-11 is used. Results computed include mean animal weight, tumor size, tumor weight, survival time. For confirmed therapeutic activity, the test composition should be tested in two multi-dose assays.

I. 3LL Lewis Lung Carcinoma: Primary Growth and Metastasis Model

This model has been utilized by a number of investigators. See, for example, Gorelik, E. et al., J Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982); Talmadge J. E. et al., J Nat'l. Canc. Inst. 69:975-980 (1982); Hilgard, P. et al., Br. J. Cancer 35:78-86(1977)). Test mice are male C57BL/6 mice, 2-3 months old. Following sc, im, or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725).

Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 3-4 days after dorsal sc injection of 10⁶ cells. The day of tumor appearance and the diameters of established tumors are measured by caliper every two days.

The treatment is given as one or two doses of peptide or derivative, per week. In another embodiment, the peptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M. L. et al., J. Lab. Clin. Med. 89:217-228 (1977). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd (iododeoxyuridine). One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints; or (2) mice are left intact as nonamputated tumor-bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

For a compound to be useful in accordance with this invention, it should demonstrate activity in at least one of the above (in vitro or in vivo) assay systems.

Pharmaceutical and Therapeutic Compositions and Their Administration

The compounds that may be employed in the pharmaceutical compositions of the invention include all of the polypeptide and peptide compounds described above, as well as the pharmaceutically acceptable salts of these compounds. Pharmaceutically acceptable acid addition salts of the compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts.

Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

As stated above, the compounds of the invention possess the ability to inhibit endothelial cell proliferation, motility, or invasiveness and angiogenesis, properties that are exploited in the treatment of cancer, in particular metastatic cancer. A composition of this invention may be active per se, or may act as a “pro-drug” that is converted in vivo to the active form.

Therapeutically Labeled Compositions

In a preferred embodiment, the polypeptide and peptides describe herein are “therapeutically conjugated” or “therapeutically labeled” (terms which are intended to be interchangeable) and used to deliver a therapeutic agent to the site to which the compounds home and bind, such as sites of tumor metastasis or foci of infection/inflammation, restenosis or fibrosis. The term “therapeutically conjugated” means that the modified peptide is conjugated to another therapeutic agent that is directed either to the underlying cause or to a “component” of tumor invasion, angiogenesis, inflammation or other pathology. A therapeutically labeled protein or peptide carries a suitable therapeutic “label” also referred to herein as a “therapeutic moiety.” A therapeutic moiety is an atom, a molecule, a compound or any chemical component added to the peptide that renders it active in treating a target disease or condition, primarily one a associated with undesired angiogenesis. As noted above, the peptides of the present invention are prepared by conventional means, either chemical synthesis, proteolysis of HPRG or recombinant means. The therapeutic moiety may be bound directly or indirectly to the peptide. The therapeutically labeled protein or peptide is administered as pharmaceutical composition which comprises a pharmaceutically acceptable carrier or excipient, and is preferably in a form suitable for injection.

Examples of useful therapeutic radioisotopes (ordered by atomic number) include ⁴⁷Sc, ⁶⁷Cu, ⁹⁰Y, ¹⁰⁹Pd, ¹²⁵I, ¹⁸⁶Re, ¹⁸⁸Re, ¹⁹⁹Au, ²¹¹At, ²¹²Pb and ²¹⁷Bi. These atoms can be conjugated to the peptide directly, indirectly as part of a chelate, or, in the case of iodine, indirectly as part of an iodinated Bolton-Hunter group. The radioiodine can be introduced either before or after this group is coupled to the peptide compound.

Preferred doses of the radionuclide conjugates are a function of the specific radioactivity to be delivered to the target site which varies with tumor type, tumor location and vascularization, kinetics and biodistribution of the peptide carrier, energy of radioactive emission by the nuclide, etc. Those skilled in the art of radiotherapy can readily adjust the dose of the peptide in conjunction with the dose of the particular nuclide to effect the desired therapeutic benefit without undue experimentation.

Another therapeutic approach included here is the use of boron neutron capture therapy, where a boronated peptide is delivered to a desired target site, such as a tumor, most preferably an intracranial tumor (Barth, R. F., Cancer Invest. 14:534-550 (1996); Mishima, Y. (ed.), Cancer Neutron Capture Therapy, New York: Plenum Publishing Corp., 1996; Soloway, A. H., et al., (eds), J. Neuro-Oncol. 33:1-188 (1997). The stable isotope ¹⁰B is irradiated with low energy (<0.025 eV) thermal neutrons, and the resulting nuclear capture yields α-particles and ⁷Li nuclei which have high linear energy transfer and respective path lengths of about 9 and 5 μm. This method is predicated on ¹⁰B accumulation in the tumor with lower levels in blood, endothelial cells and normal tissue (e.g., brain). Such delivery has been accomplished using epidermal growth factor (Yang. W. et al., Cancer Res 57:4333-4339 (1997).

Other therapeutic agents which can be coupled to the peptide compounds according to the method of the invention are drugs, prodrugs, enzymes for activating pro-drugs, photosensitizing agents, nucleic acid therapeutics, antisense vectors, viral vectors, lectins and other toxins.

Lectins are proteins, commonly derived from plants, that bind to carbohydrates. Among other activities, some lectins are toxic. Some of the most cytotoxic substances known are protein toxins of bacterial and plant origin (Frankel, A. E. et al., Ann. Rev. Med. 37:125-142 (1986)). These molecules binding the cell surface and inhibit cellular protein synthesis. The most commonly used plant toxins are ricin and abrin; the most commonly used bacterial toxins are diphtheria toxin and Pseudomonas exotoxin A. In ricin and abrin, the binding and toxic functions are contained in two separate protein subunits, the A and B chains. The ricin B chain binds to the cell surface carbohydrates and promotes the uptake of the A chain into the cell. Once inside the cell, the ricin A chain inhibits protein synthesis by inactivating the 60S subunit of the eukaryotic ribosome Endo, Y. et al., J. Biol. Chem. 262: 5908-5912 (1987)). Other plant derived toxins, which are single chain ribosomal inhibitory proteins, include pokeweed antiviral protein, wheat germ protein, gelonin, dianthins, momorcharins, trichosanthin, and many others (Strip, F. et al., FEBS Lett. 195:1-8 (1986)). Diphtheria toxin and Pseudomonas exotoxin A are also single chain proteins, and their binding and toxicity functions reside in separate domains of the same protein Pseudomonas exotoxin A has the same catalytic activity as diphtheria toxin. Ricin has been used therapeutically by binding its toxic α-chain, to targeting molecules such as antibodies to enable site-specific delivery of the toxic effect. Bacterial toxins have also been used as anti-tumor conjugates. As intended herein, a toxic peptide chain or domain is conjugated to a compound of this invention and delivered in a site-specific manner to a target site where the toxic activity is desired, such as a metastatic focus. Conjugation of toxins to protein such as antibodies or other ligands are known in the art (Olsnes, S. et al., Immunol. Today 10:291-295 (1989); Vitetta, E. S. et al., Ann. Rev. Immunol. 3:197-212 (1985)).

Cytotoxic drugs that interfere with critical cellular processes including DNA, RNA, and protein synthesis, have been conjugated to antibodies and subsequently used for in vivo therapy. Such drugs, including, but not limited to, daunorubicin, doxorubicin, methotrexate, and Mitomycin C are also coupled to the compounds of this invention and used therapeutically in this form.

The compounds of the invention, as well as the pharmaceutically acceptable salts thereof, may be incorporated into convenient dosage forms, such as capsules, impregnated wafers, tablets or injectable preparations. Solid or liquid pharmaceutically acceptable carriers may be employed.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pa. (Gennaro 18th ed. 1990).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the diagnosis or treatment of any of a number of animal genera and species, and are equally applicable in the practice of human or veterinary medicine. Thus, the pharmaceutical compositions can be used to treat domestic and commercial animals, including birds and more preferably mammals, as well as humans.

The term “systemic administration” refers to administration of a composition or agent such as the polypeptide, peptides or nucleic acids described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial(or lung instillation), intracranial, intra-aural or intraocular. The term “local administration” refers to administration of a composition or drug into a limited, or circumscribed, anatomic space, such as intratumoral injection into a tumor mass, subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration. Injectables or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, e.g., as an ointment, cream or gel; orally; rectally; e.g., as a suppository.

For topical application, the compound may be incorporated into topically applied vehicles such as a salve or ointment. The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Preferred vehicles for non-sprayable topical preparations include ointment bases, e.g., polyethylene glycol-1000 (PEG-1000); conventional creams such as HEB cream; gels; as well as petroleum jelly and the like.

Also suitable for topic application as well as for lung instillation are sprayable aerosol preparations wherein the compound, preferably in combination with a solid or liquid inert carrier material, is packaged in a squeeze bottle or in admixture with a pressurized volatile, normally gaseous propellant. The aerosol preparations can contain solvents, buffers, surfactants, perfumes, and/or antioxidants in addition to the compounds of the invention.

For the preferred topical applications, especially for humans, it is preferred to administer an effective amount of the compound to an affected area, e.g., skin surface, mucous membrane, eyes, etc. This amount will generally range from about 0.001 mg to about 1 g per application, depending upon the area to be treated, the severity of the symptoms, and the nature of the topical vehicle employed.

Other pharmaceutically acceptable carriers for polypeptide or nucleic acid compositions of the present invention are liposomes, pharmaceutical compositions in which the active protein is contained either dispersed or variously present in corpuscles consisting of aqueous concentric layers adherent to lipidic layers. The active polypeptide or peptide, or the nucleic acid is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Therapeutic compositions for treating tumors and cancer may comprise, in addition to the peptide, one or more additional anti-tumor agents, such as mitotic inhibitors, e.g., vinblastine; alkylating agents, e.g., cyclophosphamide; folate inhibitors, e.g., methotrexate, piritrexim or trimetrexate; antimetabolites, e.g., 5-fluorouracil and cytosine arabinoside; intercalating antibiotics, e.g., adriamycin and bleomycin; enzymes or enzyme inhibitors, e.g., asparaginase, topoisomerase inhibitors such as etoposide; or biological response modifiers, e.g., interferons or interleukins. In fact, pharmaceutical compositions comprising any known cancer therapeutic in combination with the peptides disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

As discussed above, antibodies specific for epitopes of the H/P domain, by inhibiting the anti-angiogenic effects of HPRG via the H/P domain, are useful in the induction of neovascularization and can be used to treat diseases or conditions in which increased angiogenesis is desired. Such conditions include coronary artery disease and peripheral artery disease, in which therapeutic angiogenesis is know to be beneficial (Freedman S B and Isner J M, Ann Intern Med, 2002, 136:54-71 and J Mol Cell Cardiol, 2001 33:379-393; Durairaj, A. et al., Cardiol Rev, 2000, 8:279-287; Emanueli C et al., Br J Pharmacol, 2001, 133:951-958; Isner, J M et al., Hum Gene Ther, 1996, 7:959-88). In general, any form of tissue ischemia resulting from vascular occlusion, vascular disease or surgery can be treated in this manner (Isner et al., supra; Webster K A., Crit Rev Eukaryot Gene Expr, 2000, 10:113-125), for example peripheral limb ischemia or hepatic arterial occlusion in liver transplantation (Yedlicka, J W et al., J Vasc Interv Radiol, 1991, 2:235-240) where the present antibodies will promote revascularization of ischemic tissues.

These antibodies are useful in the promotion of wound healing (including recovery from surgical wounds), which is known to be dependent upon angiogenic processes (Liekens S et al., Biochem Pharmacol, 2001, 61:253-270; Lingen, M W, Arch Pathol Lab Med, 2001, 125:67-71; Raza S L et al., J Investig Dermatol Symp Proc, 2000, 5:47-54; Tonnesen MG et al., J Investig Dermatol Synip Proc, 2000, 5:40-46; Hunt T K, Adv Skin Wound Care, 2000, 13(2 Suppl):6-11; Grant DS et al., Adv Exp Med Biol, 2000, 476:139-154; Drixler T A et al., Eur J Surg, 2000, 166:435-446; Singer A J et al., N Engl J Med, 1999, 341:738-746; Martin, P, Science, 1997, 276:75-81) and in accelerating or enhancing fracture repair (Glowacki, J, Clin Orthop, 1998, 355 Suppl:S82-89).

Anti-H/P antibodies can be used in conjunction with cellular therapy and transplantation of pancreatic islet cells in the treatment of diabetes as vascular endothelium acts to stimulate or induce pancreatic organogenesis and insulin production by pancreatic beta cells (Lammert E et al., Science, 2001, 294:564-567; see also page 530-531). Liver organogenesis is also promoted by vasculogenic endothelial cells and nascent vessels (Matsumoto, K. et al., Science, 2001, 294:559-563). See also, DeFrancesco, L., The Scientist 15:17 (2001).

Screening of antibodies or supernatants of hybridoma cultures to detect anti-H/P antibodies with the desired pro-angiogenic activity are performed using the in vitro and in vivo bioassays described above, such as the Matrigel® plug assay.

Therapeutic Methods

The methods of this invention may be used to inhibit tumor growth and invasion in a subject or to suppress angiogenesis induced by tumors by inhibiting endothelial cell growth and migration. By inhibiting the growth or invasion of a tumor or angiogenesis, the methods result in inhibition of tumor metastasis. A vertebrate subject, preferably a mammal, more preferably a human, is administered an amount of the compound effective to inhibit tumor growth, invasion or angiogenesis. The compound or pharmaceutically acceptable salt thereof is preferably administered in the form of a pharmaceutical composition as described above.

Doses of the proteins (including antibodies), peptides, peptide multimers, etc., preferably include pharmaceutical dosage units comprising an effective amount of the peptide. Dosage unit form refers to physically discrete units suited as unitary dosages for a mammalian subject; each unit contains a predetermined quantity of active material (e.g., the HPRG-derived domain or peptide, or nucleic acid encoding the polypeptide) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

By an effective amount is meant an amount sufficient to achieve a steady state concentration in vivo which results in a measurable reduction in any relevant parameter of disease and may include growth of primary or metastatic tumor, any accepted index of inflammatory reactivity, or a measurable prolongation of disease-free interval or of survival. For example, a reduction in tumor growth in 20% of patients is considered efficacious (Frei III, E., The Cancer Journal 3:127-136 (1997)). However, an effect of this magnitude is not considered to be a minimal requirement for the dose to be effective in accordance with this invention.

In one embodiment, an effective dose is preferably 10-fold and more preferably 100-fold higher than the 50% effective dose (ED₅₀) of the compound in an in vivo assay as described herein.

The amount of active compound to be administered depends on the precise peptide or derivative selected, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

A preferred dose for treating a subject, preferably mammalian, more preferably human, with a tumor is an amount of up to about 100 milligrams of active protein or peptide-based compound per kilogram of body weight. A typical single dosage of the peptide or peptidomimetic is between about 1 ng and about 100 mg/kg body weight. For topical administration, dosages in the range of about 0.01-20% concentration (by weight) of the compound, preferably 1-5%, are suggested. A total daily dosage in the range of about 0.1 milligrams to about 7 grams is preferred for intravenous administration. The foregoing ranges are, however, suggestive, as the number of variables in an individual treatment regime is large, and considerable excursions from these preferred values are expected.

An effective amount or dose of the peptide for inhibiting endothelial cell proliferation or migration in vitro is in the range of about 1 picogram to about 5 nanograms per cell. Effective doses and optimal dose ranges may be determined in vitro using the methods described herein.

The compounds of the invention may be characterized as producing an inhibitory effect on tumor cell or endothelial cell proliferation, migration, invasion, or on angiogenesis, on tumor metastasis or on inflammatory reactions. The compounds are especially useful in producing an anti-tumor effect in a mammalian host, preferably human, harboring a tumor.

Angiogenesis inhibitors may play a role in preventing inflammatory angiogenesis and gliosis following traumatic spinal cord injury, thereby promoting the reestablishment of neuronal connectivity (Wamil, A. W. et al., Proc. Nat'l. Acad. Sci. USA 95:13188-13193 (1998)). Therefore, the compositions of the present invention are administered as soon as possible after traumatic spinal cord injury and for several days up to about two weeks thereafter to inhibit the angiogenesis and gliosis that would sterically prevent reestablishment of neuronal connectivity. The treatment reduces the area of damage at the site of spinal cord injury and facilitates regeneration of neuronal function and thereby prevents paralysis. The compounds of the invention are expected also to protect axons from Wallerian degeneration, reverse aminobutyrate-mediated depolarization (occurring in traumatized neurons), and improve recovery of neuronal conductivity of isolated central nervous system cells and tissue in culture.

General Recombinant DNA Methods

Basic texts disclosing general methods of molecular biology, all of which are incorporated by reference, include: Sambrook, J et al., Molecular Cloning: A Laboratory Manual, 2nd (or later) Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, editor, DNA Cloning: A Practical Approach, vol. I & II, IRL Press, 1985; Albers, B. et al., Molecular Biology of the Cell, 2nd (or later) Ed., Garland Publishing, Inc., New York, N.Y. (1989); Watson, J D et al., Recombinant DNA, 2nd (or later) Ed., Scientific American Books, New York, 1992; and Old, R W et al., Principles of Gene Manipulation: An Introduction to Genetic Engineering, 2nd (or later) Ed., University of California Press, Berkeley, Calif. (1981).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, cDNA molecules encoding the amino acid sequence corresponding to the HPRG polypeptide, domain or peptide fragment of the present invention, or active variants thereof, can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

This invention includes isolated nucleic acids having a nucleotide sequence encoding the novel HPRG polypeptide, domain, peptide fragment, peptide multimer, or equivalents thereof, and their use in transfecting cells in vitro or in vivo to express their polypeptide product. The term nucleic acid as used herein is intended to include such fragments or equivalents. The nucleic acid sequences of this invention can be DNA or RNA.

A cDNA nucleotide sequence an HPRG polypeptide can be obtained by isolating total mRNA from an appropriate cell line. Double stranded cDNA is prepared from total mRNA. cDNA can be inserted into a suitable plasmid, bacteriophage or viral vector using any one of a number of known techniques.

In reference to a nucleotide sequence, the term “equivalent” is intended to include sequences encoding structurally homologous and/or a functionally equivalent proteins such as naturally occurring isoforms or related, immunologically cross-reactive family members of these proteins. Such isoforms or family members are defined as proteins that share function and amino acid sequence similarity to, for example, SEQ ID NO: 1, 3, 5 or 6.

Fragments of Nucleic Acid

A fragment of the nucleic acid sequence is defined as a nucleotide sequence having fewer nucleotides than the nucleotide sequence encoding the full length HPRG protein or H/P domain. This invention includes such nucleic acid fragments that encode polypeptides which retain (1) the ability of the HPRG polypeptide to inhibit angiogenesis, endothelial tube formation, cell invasion or tumor growth or metastasis.

Generally, the nucleic acid sequence encoding a fragment of HPRG comprises of nucleotides from the sequence encoding the mature protein (or the active H/P domain thereof).

Nucleic acid sequences, particularly those that encode peptide multimers of this invention may also include linker or spacer sequences (preferably encoding Gly1-6). The nucleic acids further may include natural or modified restriction endonuclease sites and other sequences that are useful for manipulations related to cloning, expression or purification of encoded polypeptide or peptides. These and other modifications of nucleic acid sequences are described herein or are well-known in the art.

The techniques for assembling and expressing DNA coding sequences include synthesis of oligonucleotides, PCR, transforming cells, constructing vectors, expression systems, and the like; these are well-established in the art such that those of ordinary skill are familiar with standard resource materials, specific conditions and procedures.

Expression Vectors and Host Cells

This invention includes an expression vector comprising a nucleic acid sequence encoding a HPRG polypeptide, domain, peptide or peptide multimer operably linked to at least one regulatory sequence.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and includes both the expression and nonexpression plasmids. Where a recombinant microorganism or cell culture is a host for an “expression vector,” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and the nature (e.g., size) of the polypeptide to be expressed.

The present expression vectors comprise the full range of nucleic acid molecules encoding the various embodiments of the HPRG polypeptide, domain or peptide fragment and its including peptide multimers, variants, etc.

Such expression vectors are used to transfect host cells (in vitro, ex vivo or in vivo) for expression of the DNA and production of the encoded proteins which include fusion proteins or peptides. It will be understood that a genetically modified cell expressing the HPRG polypeptide, domain, peptide fragment or multimer, may transiently express the exogenous DNA for a time sufficient for the cell to be useful for its stated purpose.

Host cells may also be transfected with one or more expression vectors that singly or in combination comprise DNA encoding at least a portion of the HPRG polypeptide or H/P, domain and DNA encoding at least a portion of a second HPRG-derived sequence (or variant), so that the host cells produce yet further HPRG polypeptide, domain or peptide fragments that include both the portions.

Methods for producing the HPRG polypeptide, domain or peptide fragments, are all conventional in the art. Cultures typically includes host cells, appropriate growth media and other byproducts. Suitable culture media are well known in the art. The HPRG polypeptide, domain or peptide fragment can be isolated from medium or cell lysates using conventional techniques for purifying proteins and peptides, including ammonium sulfate precipitation, fractionation column chromatography (e.g. ion exchange, gel filtration, affinity chromatography, etc.) and/or electrophoresis (see generally, Meth Enzymol, 22:233-577 (1971)). Once purified, partially or to homogeneity, the recombinant polypeptides of the invention can be utilized in pharmaceutical compositions as described in more detail herein.

The term “isolated” as used herein, when referring to a molecule or composition, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). It is understood that even where a protein has been isolated so as to appear as a homogenous or dominant band in a gel pattern, there are generally trace contaminants which co-purify with it.

Prokaryotic or eukaryotic host cells transformed or transfected to express the HPRG polypeptide, domain or peptide fragment are within the scope of the invention. For example, the HPRG polypeptide, domain or peptide fragment may be expressed in bacterial cells such as E. coli, insect cells (baculovirus), yeast, or mammalian cells such as Chinese hamster ovary cells (CHO) or human cells (which are preferred for human therapeutic use of the transfected cells). Other suitable host cells may be found in Goeddel, (1990) supra or are otherwise known to those skilled in the art.

Expression in eukaryotic cells leads to partial or complete glycosylation and/or formation of relevant inter- or intra-chain disulfide bonds of the recombinant polypeptide.

Examples of vectors for expression in yeast S. cerevisiae include pYepSecl (Baldari et al., (1987) EMBO J. 6:229-234), pMFa (Kurjan et al. (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165,) and the pVL series (Lucklow, V. A., and Summers, M. D., (1989) Virology 170:31-39). Generally, COS cells (Gluzman, Y., (1981) Cell 23:175-182) are used in conjunction with such vectors as pCDM 8 (Aruffo A. and Seed, B., supra, for transient amplification/expression in mammalian cells, while CHO (dhfr-negative CHO) cells are used with vectors such as pMT2PC (Kaufman et al. (1987), EMBO J. 6:187-195) for stable amplification/expression in mammalian cells. The NSO myeloma cell line (a glutamine synthetase expression system.) is available from Celltech Ltd.

Often, in fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant polypeptide.

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lacO fusion promoter mediated by coexpressed viral RNA polymerase (T7gn1). Th is viral polymerase is supplied by host strains BL21(DE3) or HMS174(DE3) from a resident X prophage harboring a T7gn1 under the transcriptional control of the lacUV 5 promoter.

Vector Construction

Construction of suitable vectors containing the desired coding and control sequences employs standard ligation and restriction techniques which are well understood in the art. Isolated plasmids, DNA sequences, or synthesized oligonucleotides are cleaved, tailored, and re-ligated in the form desired. The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotidcs and filling in single stranded nonoverlapping portions using DNA polymerase in the presence of the deoxyribonucleotide triphosphates. This approach has been used successfully in the construction of several genes of known sequence. See, for example, Edge, M. D., Nature (1981) 292:756; Nambair, K. P., et al., Science (1984) 223:1299; and Jay, E., J. Biol Chem (1984) 259:6311.

Synthetic oligonucleotides are prepared by either the phosphotriester method as described by references cited above or the phosphoramidite method as described by Beaucage, S. L., and Caruthers, M. H., Tetrahed Lett (1981) 22:1859; and Matteucci, M. D., and Caruthers, M. H., J Am Chem Soc (1981) 103:3185 and can be prepared using commercially available automated oligonucleotide synthesizers. Kinase treatment of single strands prior to annealing or for labeling is achieved using well-known methods.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. If desired, size separation of the cleaved fragments may be performed by polyacrylamide gel or agarose gel electrophoresis using standard techniques. A general description of size separations is found in Meth Enzymol (1980) 65:499-560.

Any of a number of methods are used to introduce mutations into the coding sequence to generate variants of the invention if these are to be produced recombinantly. These mutations include simple deletions or insertions, systematic deletions, insertions or substitutions of clusters of bases or substitutions of single bases. Modifications of the DNA sequence are created by site-directed mutagenesis, a well-known technique for which protocols and reagents are commercially available (Zoller, M J et al., Nucleic Acids Res (1982) 10:6487-6500 and Adelman, J P et al., DNA (1983) 2:183-193)). The isolated DNA is analyzed by restriction and/or sequenced by the dideoxy nucleotide method of Sanger (Proc Natl Acad Sci USA (1977) 74:5463) as further described by Messing, et al., Nucleic Acids Res (1981) 9:309, or by the method of Maxam et al, Meth. Enzymol., supra.

Vector DNA can be introduced into mammalian cells via conventional techniques such as calcium phosphate or calcium chloride co-precipitation, DEAE-dextran-mediated transfection, lipofection, or electroporation. Suitable methods for transfoming host cells can be found in Sambrook et al. supra and other standard texts. In fusion expression vectors, a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521 (1985)) or in the RSV-LTR (from Rous sarcoma virus) (Gorman, C. M., Proc. Natl. Acad. Sci. USA 79:6777 (1982). Also useful are the promoter of the mouse metallothionein I gene (Hamer, D., et al., J. Mol. Appl. Gen. 1:273-288 (1982)); the TK promoter of Herpes virus (McKnight, S., Cell 31:355-365 (1982)); the SV40 early promoter (Benoist, C., et al., Nature 290:304-310 (1981)); and the yeast gal4 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)). Other illustrative descriptions of transcriptional factor association with promoter regions and the separate activation and DNA binding of transcription factors include: Keegan et al., Nature (1986) 231:699; Fields et al., Nature (1989) 340:245; Jones, Cell (1990) 61:9; Lewin, Cell (1990) 61:1161; Ptashne et al., Nature (1990) 346:329; Adams et al., Cell (1993) 72:306. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B. M., Genes IV, Oxford University Press, Oxford, (1990), pp. 552-576. Particularly useful are retroviral enhancers (e.g., viral LTR). The enhancer is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

The nucleic acid sequences of the invention can also be chemically synthesized using standard techniques. Various methods of chemically synthesizing polydeoxynucleotides are known, including solid-phase synthesis which, like peptide synthesis, has been fully automated with commercially available DNA synthesizers (See, e.g., Itakura et al. U.S. Pat. No. 4,598,049; Caruthers et al U.S. Pat. No. 4,458,066; and Itakura U.S. Pat. Nos. 4,401,796 and 4,373,071, incorporated by reference herein).

Delivery of Nucleic Acid to Cells and Animals

DNA delivery involves introduction of a “foreign” DNA either (1) into a cell ex vivo and ultimately, into a live animal by administering the cells, or (2) directly into the animal. Several general strategies for “gene delivery” (i.e., delivery of any nucleic acid vector) for purposes that include “gene therapy” have been studied and reviewed extensively (Yang, N-S., Crit. Rev. Biotechnol. 12:335-356 (1992); Anderson, W. F., Science 256:808-813 (1992); Miller, A. S., Nature 357:455-460 (1992); Crystal, R. G., Amer. J. Med. 92(suppl 6A):44S-52S (1992); Zwiebel, J. A. et al., Ann. N.Y. Acad. Sci. 618:394-404 (1991); McLachlin, J. R. et al., Prog. Nucl. AcidRes. Molec. Biol. 38:91-135 (1990); Kohn, D. B. et al., Cancer Invest. 7:179-192 (1989), which references are herein incorporated by reference in their entirety).

One approach comprises nucleic acid transfer into primary cells in culture followed by autologous transplantation of the ex vivo transformed cells into the host, either systemically or into a particular organ or tissue.

Preferred DNA molecules for delivery as described below encode HPRG, e.g., SEQ ID NO: 1 or 3, the H/P domain thereof (SEQ ID NO:5 or 6) or peptides or peptide multimers based on SEQ ID NO:7, 8, 9 or 10.

For accomplishing the objectives of the present invention, nucleic acid therapy would be accomplished by direct transfer of a the functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Examples of successful “gene transfer” reported in the art include: (a) direct injection of plasmid DNA into mouse muscle tissues, which led to expression of marker genes for an indefinite period of time (Wolff, J. A. et al., Science 247:1465 (1990); Acsadi, G. et al., The New Biologist 3:71 (1991)); (b) retroviral vectors are effective for in vivo and in situ infection of blood vessel tissues; (c) portal vein injection and direct injection of retrovirus preparations into liver effected gene transfer and expression in vivo (Horzaglou, M. et al., J. Biol. Chem. 265:17285 (1990); Koleko, M. et al., Human Gene Therapy 2:27 (1991); Ferry, N. et al., Proc. Natl. Acad. Sci. USA 88:8387 (1991)); (d) intratracheal infusion of recombinant adenovirus into lung tissues was effective for in vivo transfer and prolonged expression of foreign genes in lung respiratory epithelium (Rosenfeld, M. A. et al., Science 252:431 (1991); (e) Herpes simplex virus vectors achieved in vivo gene transfer into brain tissue (Ahmad, F. et al., eds, Miami Short Reports—Advances in Gene Technology: The Molecular Biology of Human Genetic Disease, Vol 1, Boehringer Manneheiml Biochemicals, USA, 1991). Gene therapy of cystic fibrosis using transfection by plasmids using any of a number of methods and by retroviral vectors has been described by Collins et al., U.S. Pat. No. 5,240,846.

Retroviral-mediated human therapy utilizes amphotrophic, replication-deficient retrovirus systems (Temin, H. M., Human Gene Therapy 1:111 (1990); Temin et al., U.S. Pat. No. 4,980,289; Temin et al., U.S. Pat. No. 4,650,764; Temin et al., U.S. Pat. No. 5,124,263; Wills, J. W. U.S. Pat. No. 5,175,099; Miller, A. D., U.S. Pat. No. 4,861,719). Such vectors have been used to introduce functional DNA into human cells or tissues, for example, the adenosine deaminase gene into lymphocytes, the NPT-II gene and the gene for tumor necrosis factor into tumor infiltrating lymphocytes. Retrovirus-mediated gene delivery generally requires target cell proliferation for gene transfer (Miller, D. G. et al., Mol. Cell. Biol. 10:4239 (1990). This condition is met by certain of the preferred target cells into which the present DNA molecules are to be introduced, i.e., actively growing tumor cells. The DNA molecules encoding the HPRG polypeptide, domain or peptide fragments of the present invention may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R. F. et al., Cell 33:153-159 (1983); Miller, A. D. et al., Molec. Cell. Biol. 5:431-437 (1985),; Sorge, J., et al., Molec. Cell. Biol. 4:1730-1737 (1984); Hock, R. A. et al., Nature 320:257 (1986); Miller, A. D. et al., Molec. Cell. Biol. 6:2895-2902 (1986). Newer packaging cell lines which are efficient an safe for gene transfer have also been described (Bank et al., U.S. Pat. No. 5,278,056.

This approach can be utilized in a site specific manner to deliver the retroviral vector to the tissue or organ of choice. Thus, for example, a catheter delivery system can be used (Nabel, EG et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M. S., In: Virology, Fields, BN et al., eds, Raven Press, New York, 1990, p. 1679; Berkner, K. L., Biotechniques 6:616 9191988), Strauss, S. E., In: The Adenoviruses, Ginsberg, H S, ed., Plenum Press, New York, 1984, chapter 11), herpes simplex virus (HSV) for neuron-specific delivery and persistence. Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R. J. et al., EMBO J. 10:3941 (1991) in the present invention.

Another useful vector, particularly in humans, is vaccinia virus, which can be rendered non-replicating (U.S. Pat. Nos. 5,225,336; 5,204,243; 5,155,020; 4,769,330; Sutter, G et al., Proc. Natl. Acad. Sci. USA (1992) 89:10847-10851; Fuerst, T. R. et al., Proc. Natl. Acad. Sci. USA (1989) 86:2549-2553; Falkner F. G. et al.; Nucl. Acids Res (1987) 15:7192; Chakrabarti, S et al., Molec. Cell. Biol. (1985) 5:3403-3409). Descriptions of recombinant vaccinia viruses and other viruses containing heterologous DNA and their uses in immunization and DNA therapy are reviewed in: Moss, B., Curr. Opin. Genet. Dev. (1993) 3:86-90; Moss, B. Biotechnology (1992) 20:345-362; Moss, B., Curr Top Microbiol Immunol (1992)158:25-38; Moss, B., Science (1991) 252:1662-1667; Piccini, A et al., Adv. Virus Res. (1988) 34:43-64; Moss, B. et al., Gene Amplif Anal (1983) 3:201-213.

In addition to naked DNA or RNA, or viral vectors, engineered bacteria may be used as vectors. A number of bacterial strains including Salmonella, BCG and Listeria monocytogenes (LM) (Hoiseth & Stocker, Nature 291, 238-239 (1981); Poirier, TP et al. J. Exp. Med. 168, 25-32 (1988); (Sadoff, J. C., et al., Science 240, 336-338 (1988); Stover, C. K., et al., Nature 351, 456-460 (1991); Aldovini, A. et al., Nature 351, 479-482 (1991); Schafer, R., et al., J. Immunol. 149, 53-59 (1992); Ikonomidis, G. et al., J. Exp. Med. 180, 2209-2218 (1994)). These organisms permit enteric routes of infection, providing the possibility of oral nucleic acid delivery.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N. -S., et al., Proc. Natl. Acad. Sci. USA 87:9568 (1990); Williams, R. S. et al., Proc. Natl. Acad. Sci. USA 88:2726 (1991); Zelenin, A. V. et al., FEBS Lett. 280:94 (1991); Zelenin, A. V. et al., FEBS Lett. 244:65 (1989); Johnston, S. A. et al., In Vitro Cell. Dev. Biol. 27:11 (1991)). Furthermore, electroporation, a well-known means to transfer genes into cell in vitro, can be used to transfer DNA molecules of the present invention to tissues in vivo (Titomirov, A. V. et al., Biochim. Biophys. Acta 1088:131 ((1991)).

“Carrier mediated gene transfer” has also been described (Wu, C. H. et al., J. Biol. Chem. 264:16985 (1989); Wu, G. Y. et al., J. Biol. Chem. 263:14621 (1988); Soriano, P. et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc. Natl. Acad. Sci. USA 80:1068 (1983); Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA of the present invention for transfer.

Plasmid DNA used for transfection or microinjection may be prepared using methods well-known in the art, for example using the Quiagen procedure (Quiagen), followed by DNA purification using known methods, such as the methods exemplified herein.

Having now generally described the invention, the same will be more readily understood through reference to the following examples which are provided by way of illustration, and are not intended to be limiting of the present invention, unless specified.

### EXAMPLE I

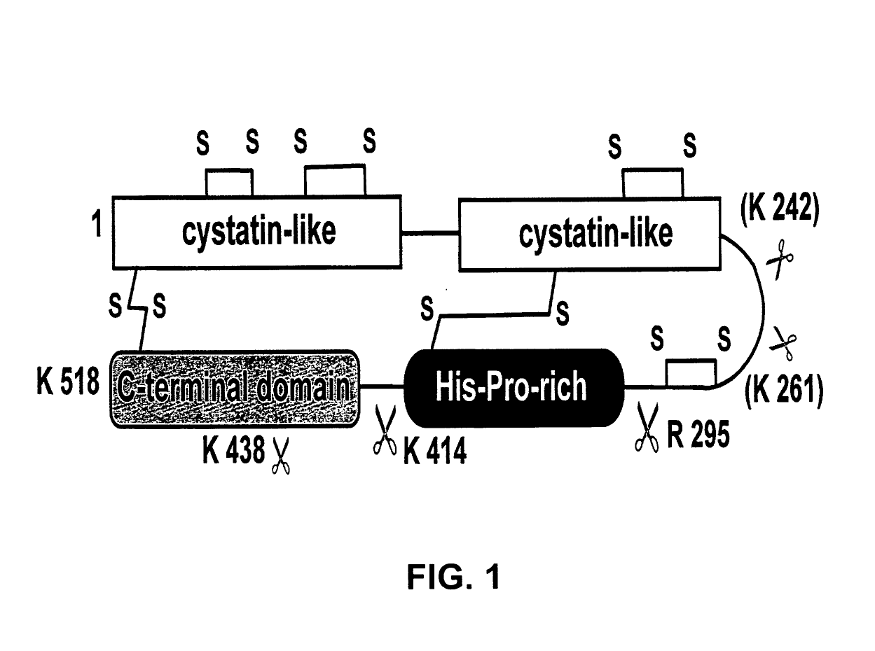

Rabbit HPRG is cleaved by plasmin to release the His-Pro-rich domain (H/P) and the residual N/C domain. The domain structure is illustrated in FIG. 1. The scissors in the Figure illustrate the positions of plasmin cleavage.

These domains can then be purified and tested in vitro and in vivo for anti-angiogenic activity in order to identify the region of HPRG that mediates anti-angiogenic effects.

### EXAMPLE II

### Inhibition of Endothelial Cell Proliferation by HPRG

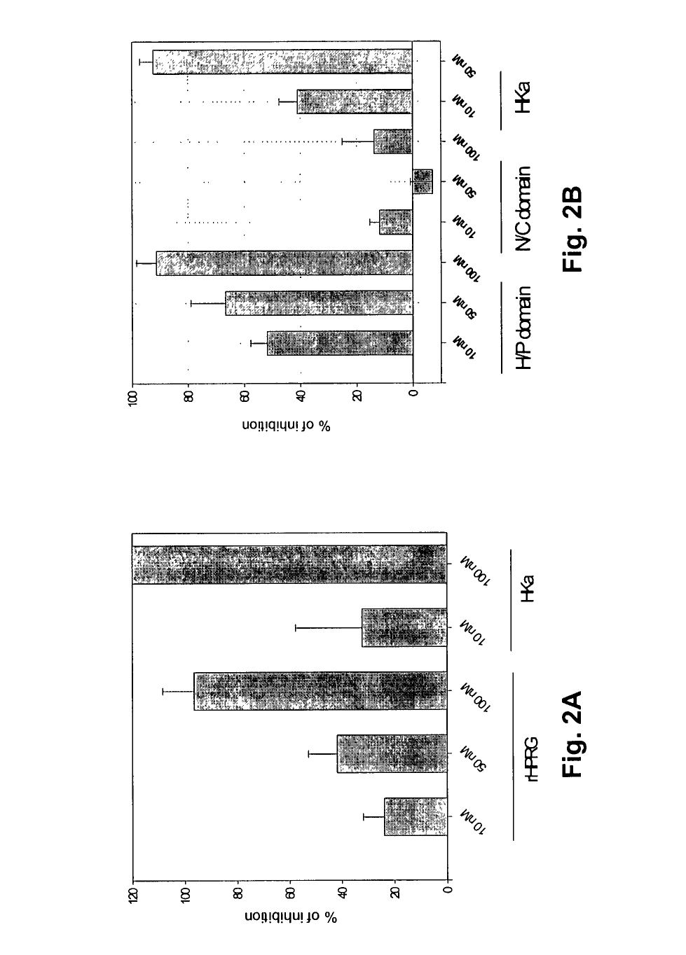

bFGF is used to stimulate human umbilical vein endothelial cell (HUVEC) proliferation. Cells are incubated in the presence of bFGF alone or with added inhibitors of proliferation for 48 hours in a 96 well plate. Proliferation is then measured using the colorimetric reagent, MTS. Results in FIGS. 2A and 2B are presented as a percentage of the proliferation observed in wells incubated with bFGF alone (100% proliferation).

Rabbit HPRG (rHPRG) inhibited bFGF-stimulated proliferation of HUVEC in a dose dependent manner, the inhibition being almost complete at 100 nM as shown in FIG. 2A.

FIG. 2B shows that the H/P domain prepared by limited proteolysis of HPRG by plasmin retained the anti-proliferative activity of intact HPRG, whereas the proteolysis product (the N/C domain, which included all the domains of HPRG but the H/P domain) had no activity. HKa is included as a positive control.

Viability of HUVEC treated with 1 μM HPRG was not less than controls, indicating that this polypeptide is not cytotoxic to HUVEC.

### EXAMPLE III

### HPRG and the H/P Domains Induce Endothelial Cell Apoptosis Through the Induction of Caspase-3 Activity

In order to evaluate whether the observed anti-proliferative activity of HPRG was due to an induction of apoptosis, the activity of caspase-3 (an enzyme that is central to several pro-apoptotic pathways) was measured. HUVEC were grown in 100 mm² petri dishes in the presence of bFGF or bFGF+HPRG. Cells were extracted and caspase-3 activity measured using a fluorescent substrate.

HKa, which had previously been shown to induce caspase-3 activity in HUVECs [Zhang et al., FASEB J(2000) 14: 2589-2600] was used as a positive control.

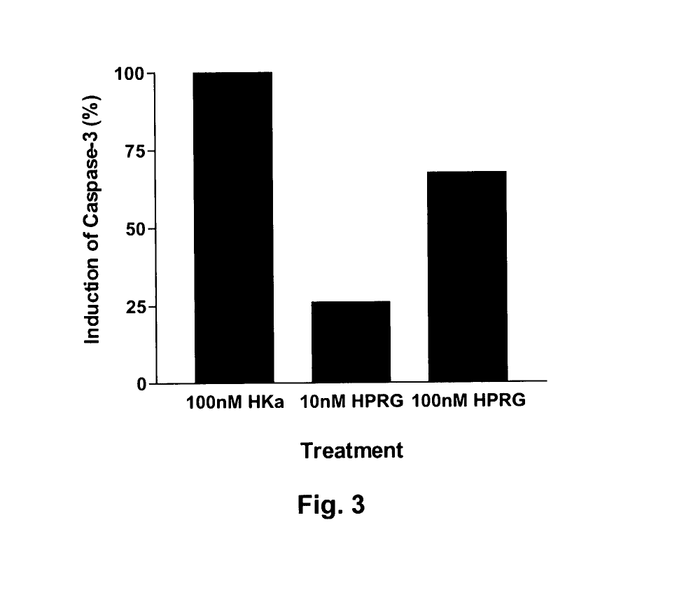

The results are shown in FIG. 3. rHPRG at 10 and 100 nM induced caspase-3 activity to a similar degree as did HKa. Results are expressed as percent of HKa activity, taken to be 100% in this assay). The H/P domain of HPRG also induced apoptosis to a similar extent at similar concentrations of H/P in the assay. This indicates that the apparent decrease in cell number observed in the proliferation assay may be related to a direct induction of cell death in endothelial cells by HPRG and its H/P domain.

### EXAMPLE IV

### Rabbit HPRG Inhibits Endothelial Cell Tube Formation of HUVECs on Matrigel®

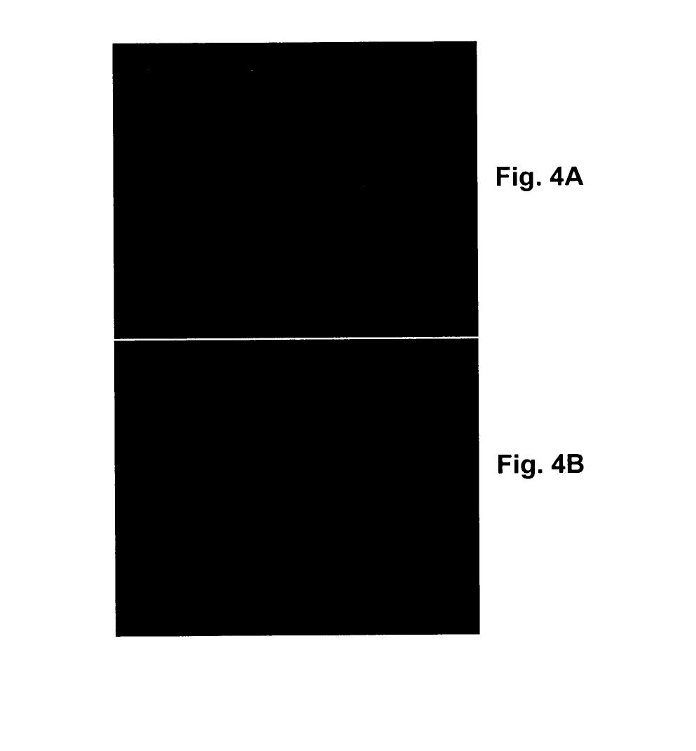

HUVEC were seeded onto Matrigel®-coated 96 well plates. Photomicrographs showing results are in FIGS. 4A and 4B.

Endothelial cell tube formation on Matrigel® was stimulated by incubation for 24 hr with FGF-2 (20 ng/ml ), VEGF (20 ng/ml ) and PMA (40 ng/ml ) for 24 hours (FIG. 4A). Addition of HPRG (500 nM ) almost completely disrupted tube formation under these conditions (FIG. 4B).

### EXAMPLE V

### HPRG (ATN-234) and the H/P (ATN-236) Domain Inhibit Angiogenesis in the CAM Model

This assay was performed essentially as described by Nguyen et al. (Microvascular Res. 47:31-40 (1994)). A filter containing either an angiogenic factor (bFGF, 30 μg/ml) or bFGF at the same concentration and an inhibitor at 20 μg/ml, was placed onto the CAM of an 8-day old chick embryo, and the CAM was observed for 3-9 days. Angiogenesis was quantitated by counting the number of microvessels that contacted the filter. In this experiment, microvessel were counted 4 days after implantation of the filter.

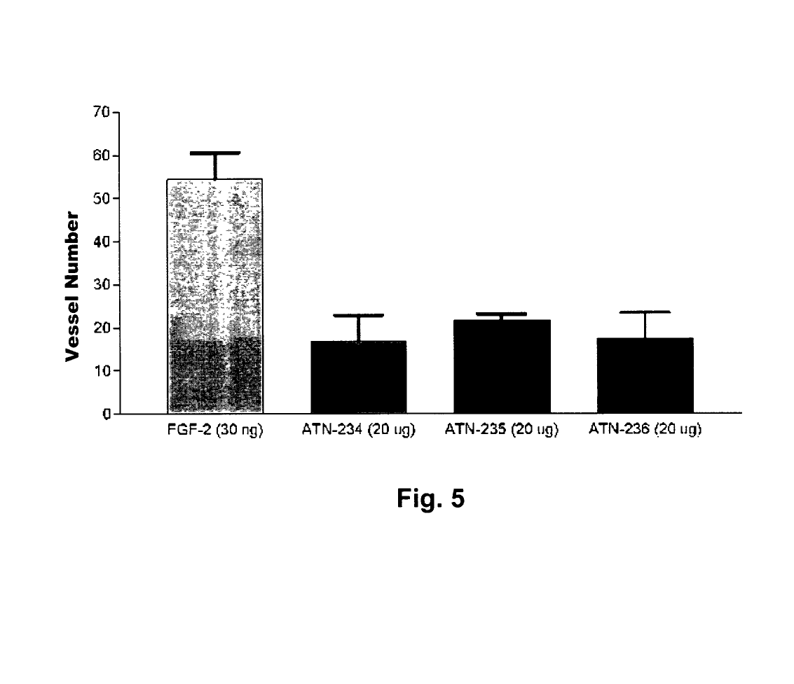

As shown in FIG. 5 HPRG (ATN-234), HKa (ATN-235) and ATN-236 (H/P domain) were all capable of inhibiting neovessel formation in this model.

### EXAMPLE VI

### HPRG and the H/P Domain Inhibit Angiogenesis Stimulated by FGF-2 in Matrigel® Plug Model in vivo

In this study, ice-cold Matrigel® (500 μL) was mixed with heparin (50 μg/ml), FGF-2 (400 ng/ml) and the compound to be tested. The Matrigel® mixture was injected subcutaneously into 4-8 week-old female Ncr athymic nude mice at sites near the abdominal midline, 3 plugs per mouse. The injected Matrigel® forms a palpable solid gel. Animals were sacrificed by cervical dislocation 7 days post injection. The mouse skin was detached along the abdominal midline, and the Matrigel® plugs were recovered and scanned immediately at high resolution. Plugs were then dispersed in water and incubated at 37° C. overnight. Hemoglobin levels were determined using Drabkin's solution (from Sigma) according to the manufacturers' instructions.

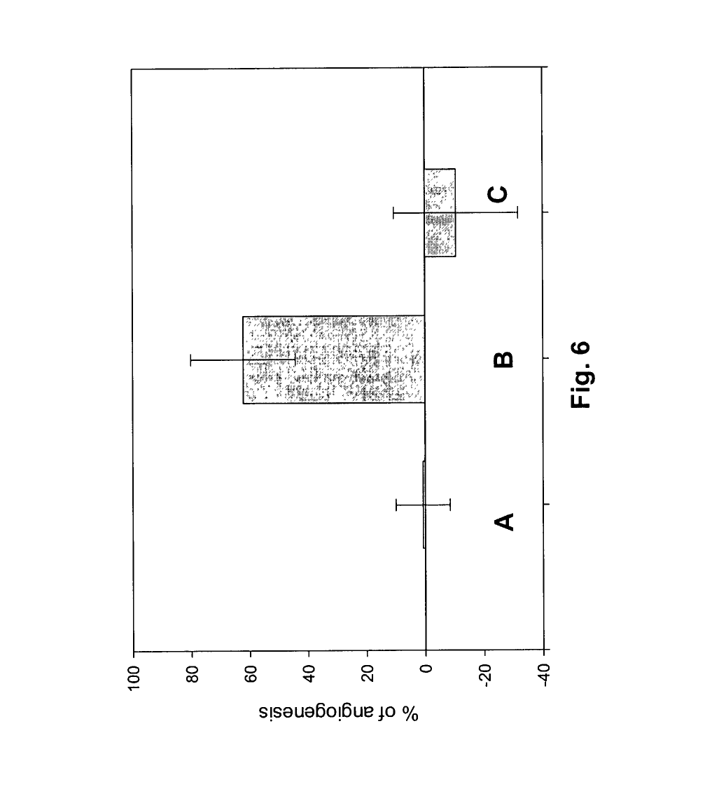

The results are shown in FIG. 6. HPRG (0.25 μM, A) and the H/P domain (0.6 μM, C) completely inhibited angiogenesis. In contrast, the N/C fragment of HPRG (0.25 μM, C) had virtually no effect on angiogenesis. The Hb level compared to the positive control was 124±42% (mean±SD of three plugs).

### EXAMPLE VII

### HPRG and the H/P Domain Inhibit Tumor Cell (3LL)-Mediated Angiogenesis in vivo in a Matrigel® Plug Model

The methods used in this study were essentially the same as described in Example VI except that 3LL tumor cells were used to stimulate angiogenesis instead of bFGF. Lewis lung adenocarcinoma 3LL cells (10⁶ cells/plug) were mixed with cold Matrigel prior to injection. After seven days, the animals are sacrificed by cervical dislocation and the Matrigel® plugs recovered and processed as above.

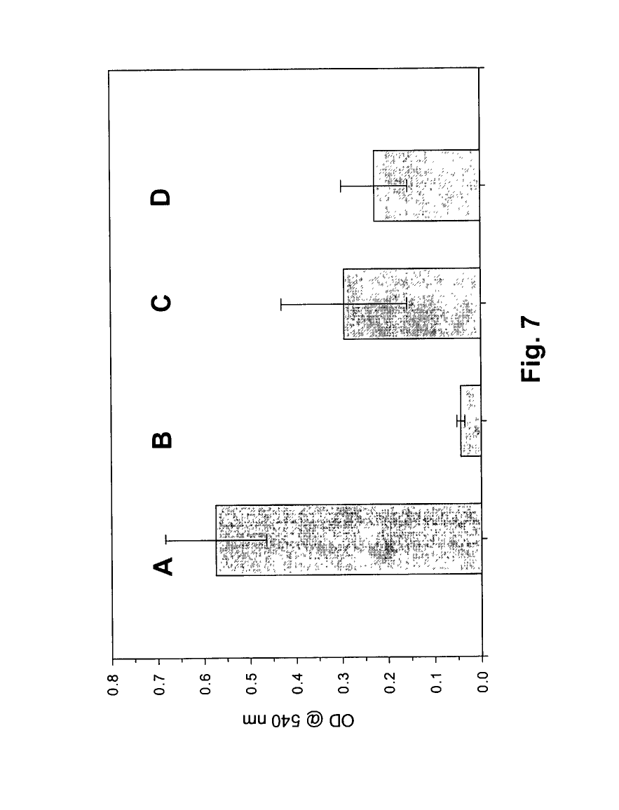

Results are shown in FIG. 7 (where amount of Hb is shown). The control group of 3LL cells alone (A) shows a maximal level of angiogenesis, whereas, in the absence of tumor cells (B), a baseline of Hb presence is observed, reflecting control levels of vascularization. A “positive” control anti-angiogenic molecule, HKa (at 0.75 μM) (C) inhibits angiogenesis by about 50%. The H/P domain of HPRG (1.8 μM) (D) shows a similar degree of inhibition.

### EXAMPLE VIII

### HPRG and the H/P Domain Inhibit Tumor Cell (MatLyLu)-mediated Angiogenesis in vivo in a Matrigel® Plug Model

The rat prostate tumor cell line (MatLyLu) was used to stimulate angiogenesis in the Matrigel® Plug model as described in Examples VII and VIII. In this study, tumor growth was evaluated.

Results are shown in FIGS. 8A and 8B. In the control group, plugs were inoculated with MatLyLu tumor cells alone. Introduction of the H/P domain (1.8 μM) together with the tumor cells resulted in a significant diminution of tumor weight (FIG. 8A) and angiogenesis (FIG. 8B). Similar effects were observed with endostatin at the same concentration.

### EXAMPLE IX

### Identification of H/P Consensus Sequences

The H/P domain (was analyzed for the presence of repeat sequences. These are described below and the and quantitated in Table 1, below. Each consensus sequence has been compared for both rabbit and human sequences.

His-Pro domain of Human HPRG (residues 350-497)

```
(SEQ ID NO:5)
350 360 370 380 390 400
| | | | | |
H PHKHHSHEQH PHGHHPHAHH PHEHDTHRQH PHGHHPGHH PHGHHPHGHH
                    410 420 430 440 450
                    | | | | |
  PHGHHPHCHD FQDYGPCDPP PHNQGHCCHG HGPPPGHLRR RGPGKGPRPF
                  460 470 480 490 497
                  | | | | |
HCRQIGSVYR LPPLRKGEVL PLPEANFPSF PLPHHKHPLK PDNQPFP
```

His-Pro domain of Rabbit HPRG (residues 321-421)

```
(SEQ ID NO:6)
321 330 340 350 360
    | | | | |
    SVNIIHRPPP HGHHPHGPPP HGHHPHGPPP HGHPPHGPPP
              370 380 390 400
                  | | | |
RHPPHGPPPH GHPPHGPPPH GHPPHGPPPH GHPPHGPPPH
              410 420
                  | |
GHPPHGHGFH DHGPCDPPSH K
```

The sequences above are annotated to show three different consensus repeats:

HHPHG (in italics) —SEQ ID NO:8

HPPHG (in double underscore)—SEQ ID NO:9

PPPHG (in single underscore)—SEQ ID NO:10

This is shown below as a different version of SEQ ID NO:6

SVNIIHR PPPHG HHPHG PPPHG HHPHG PPPHG HPPHG PPPR HPPHG PPPHG HPPHG PPPHG HPPHG PPPHG HPPHG PPPHG HPPHG GFHDHGPCDPPSHK

TABLE 1

#of repeats in

| | | | | |
|---|---|---|---|---|
| ATN#¹ | Repeated motif | SEQ ID NO: | Rabbit | Human |

| | | | | |
|---|---|---|---|---|
| ATN227 | PPPHG | 10 | 7 | 0 |
| ATN228 | HPPHG | 9 | 6 | 0 |
| ATN230 | HHPHG | 8 | 2 | 6 |

¹Applicants' company designation of compound

### EXAMPLE XI

### Consensus Sequences from the HPRG H/P Domain Inhibit Angiogenesis

Matrigel® tube formation assays in vitro were carried out as described above.

Results are summarized in Table 2, below. Two consensus sequences from the H/P domain of HPRG, HHPHG (SEQ ID NO:8) and HPPHG (SEQ ID NO:9) were active in the Matrigel® Plug assay. The N-terminal Ala-substituted variant of the latter, APPHG (SEQ ID NO: 11) had no effect on neovascularization as measured by tube formation in the Matrigel® assay.

TABLE 2

| | | | |
|---|---|---|---|
| | | | Inhibits Angiogenesis |
| ATN# | Sequence | SEQ ID NO: | (Matrigel® assay) |

| | | | |
|---|---|---|---|
| ATN230 | HHPHG | 8 | Yes |
| ATN228 | HPPHG | 9 | Yes |
| ATN246 | APPHG | 11 | No |

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. An isolated anti-angiogenic polypeptide or peptide having the sequence of
(a) the histidine-proline-rich (H/P) domain of human histidine-proline rich glycoprotein (HPRG) (SEQ ID NO:5)
(b) the H/P domain of human rabbit HPRG (SEQ ID NO:6)
(c) a sequence variant of SEQ ID NO:5 or SEQ ID NO:6 having substantially the same biologic activity of inhibiting angiogenesis, endothelial cell proliferation or endothelial tube formation in an in vitro or in vivo bioassay;
(d) a pentapeptide from said H/P domain having the sequence (His,Pro)-(His,Pro)-Pro-His-Gly (SEQ ID NO:7), or an addition variant thereof having an additional 1 to 4 amino acids selected from the group consisting of His, Pro or Gly added at the N- or C-terminus of the pentapeptide.

2. The isolated peptide of claim 1 having a sequence selected from the group consisting of His-His-Pro-His-Gly (SEQ ID NO:8), His-Pro-Pro-His-Gly (SEQ ID NO:9), or Pro-Pro-Pro-His-Gly (SEQ ID NO:10), or said addition variant thereof.

3. A chemically synthesized peptide multimer comprising the peptide or addition variant of claim 2, which multimer is selected from the group consisting of:
(a) a multimer having the formula P¹ₙ wherein
(i) P¹ is the peptide or addition variant of claim 2, and
(ii) n=2-8,
(b) a multimer having the formula (P¹-Xₘ)ₙ-P², wherein
(i) P¹ and P² are pentapeptides or addition variants according to claim,
(ii) P¹ and P² are the same or different peptides;
(iii) X is C₁-C₅ alkyl, C₁-C₅ alkenyl, C₁-C₅ alkynyl, C₁-C₅ polyether containing up to 4 oxygen atoms;
(iv) m=0 or 1; and
(v) n=1-7,
and wherein the peptide multimer has the biological activity of inhibiting angiogenesis, endothelial cell proliferation or endothelial tube formation in an in vitro or in vivo bioassay.

4. A recombinantly produced peptide multimer comprising the peptide or addition variant of claim 2, which multimer has the formula (P¹-Gly_{z})ₙ-P², wherein:
(i) P¹ and P² are pentapeptides or addition variants according to claim 2,
(ii) P¹ and P² arc the same or different;
(iii) z=0-6; and
(iv) n=1-100.

5. A diagnostically or therapeutically labeled anti-angiogenic polypeptide, peptide or peptide multimer comprising:
(a) the polypeptide, peptide or peptide multimer according to any of claims 1-4, which is diagnostically or therapeutically labeled;
(b) a diagnostically or therapeutically human HPRG protein (SEQ ID NO: 1);
(c) a diagnostically or therapeutically rabbit HPRG protein (SEQ ID NO:3); or
(d) a diagnostically or therapeutically labeled polypeptide that is a homologue of (b) or (c).,

6. The diagnostically or therapeutically labeled polypeptide or peptide of claim 5, wherein the polypeptide is selected from the group consisting of:
(a) the H/P domain of human HPRG (SEQ ID NO:5);
(b) the H/P domain of rabbit HPRG (SEQ ID NO:6); and
(c) said peptide having the sequence SEQ ID NO:7 or said addition variant thereof.

7. A diagnostically useful HPRG-related composition comprising:
(a) the diagnostically labeled polypeptide, peptide or peptide multimer of claim 5 or 6
(b) a diagnostically acceptable carrier.

8. The composition of claims 7 wherein the detectable label is a radionuclide, a PET-imageable agent, an MRI-imageable agent, a fluorescer, a fluorogen, a chromophore, a chromogen, a phosphorescer, a chemiluminescer or a bioluminescer.

9. The composition of claim 8, wherein the detectable label is a radionuclide selected from the group consisting of ³H, ¹⁴C, ³⁵S, ⁶⁷Ga, ⁶⁸Ga, ⁷²As, ⁸⁹Zr, ⁹⁷R, ⁹⁹Tc, ¹¹¹In, ¹²³I, ¹²⁵I, ¹³¹I, ¹⁶⁹Yb and ²⁰¹Tl.

10. The composition of claims 8 wherein the detectable label is a fluorescer or fluorogen selected from the group consisting of fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green and Texas Red.

11. An anti-angiogenic pharmaceutical composition comprising:
(a) an effective amount of the polypeptide, peptide or peptide multimer of any of claims 1-4; and
(b) a pharmaceutically acceptable carrier.

12. A therapeutic anti-angiogenic pharmaceutical composition comprising:
(a) an effective amount of the polypeptide, peptide or peptide multimer of claims 5 or 6 to which is bound directly or indirectly a therapeutically active moiety; and
(b) a pharmaceutically acceptable carrier.

13. The therapeutic pharmaceutical composition of claim 11 or 12 in a form suitable for injection.

14. The therapeutic pharmaceutical composition of claim 12 wherein the therapeutically active moiety is a radionuclide.

15. The therapeutic pharmaceutical composition of claim 14, wherein the radionuclide is selected from the group consisting of ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb and ²¹⁷Bi.

16. An antibody specific for an epitope of HPRG that is present in the H/P domain of human HPRG (SEQ ID NO:5) or the H/P domain of rabbit HPRG (SEQ ID NO:6), and which binds to HPRG or to any of said domains in a way which inhibits the anti-angiogenic activity of HPRG or said domain, or an antigen-binding fragment of said antibody.

17. The antibody of claim 16, wherein the epitope comprises a pentapeptide from said H/P domain having the sequence His-His-Pro-His-Gly (SEQ ID NO:8), His-Pro-Pro-His-Gly (SEQ ID NO:9), or Pro-Pro-Pro-His-Gly (SEQ ID NO: 10), or an antigen binding fragment of said antibody, which antibody or fragment inhibits the anti-angiogenic activity of said pentapeptide.

18. The antibody of claim 16 or 17 which is a monoclonal antibody.

19. The antibody of claim 18 that is a human or humanized monoclonal antibody.

20. An antibody useful for detecting HPRG comprising the antibody or fragment of any of claims 16-19, which is detectably labeled.

21. A therapeutically useful antibody that targets HPRG or an epitope thereof, comprising the antibody or fragment of any of claims 16-19 to which is bound directly or indirectly a therapeutically active moiety.

22. A pharmaceutical composition that stimulates angiogenesis in vitro or in vivo, comprising:
(a) the antibody or fragment of any of claims 16-19; and
(b) a pharmaceutically acceptable carrier.

23. A method for inhibiting cell migration, cell invasion, cell proliferation or angiogenesis, or for inducing apoptosis, comprising contacting cells associated with undesired cell migration, invasion, proliferation or angiogenesis with an effective amount of a therapeutic pharmaceutical composition according to any of claims 11-15.

24. A method for treating a subject having a disease or condition associated with undesired cell migration, invasion, proliferation, or angiogenesis, comprising administering to the subject an effective amount of a pharmaceutical composition according to any of claims 11-15.

25. A method for stimulating angiogenesis comprising providing to cells participating in angiogenesis an effective amount of the antibody or fragment of any of claims 16-19.

26. A method for stimulating angiogenesis in a subject in need of enhanced angiogenesis, comprising administering to said subject an effective amount of the pharmaceutical composition of claim 22.

27. A method for detecting the presence of HPRG or cleavage product or peptide thereof in a biological sample, comprising the steps of:
(a) contacting the sample with the antibody or fragment of claim 20; and
(b) detecting the presence of the label associated with the sample.

28. The method of claim 27 wherein the sample is plasma, serum, cells, a tissue, an organ, or an extract of said cells, tissue or organ.

29. The method of claim 27, wherein the contacting and the detecting are in vitro.

30. The method of claim 27 wherein the contacting is in vivo and the detecting is in vitro.

31. The method of claim 27 wherein the contacting is in vivo and the detecting is in vitro.

32. The method of claim 32, wherein the contacting and the detecting are in vivo.

33. An isolated nucleic acid that encodes the polypeptide or peptide of claim 1 or 2 or encodes the peptide multimer of claim 4.

34. An expression vector comprising the nucleic acid of claim 33 operatively linked to
(a) a promoter and
(b) optionally, additional regulatory sequences that regulate expression of said nucleic acid in a eukaryotic cell.

35. The expression vector of claim 34 which is a plasmid.

36. The expression vector of claim 34 which is a viral vector.

37. A cell transformed or transfected with the nucleic acid molecule of claim 33.

38. A cell transformed or transfected with the expression vector of any of claims 13-16.

39. The cell of any of claims 37 or 38 which is a mammalian cell.

40. The cell of claim 39 which is a human cell.

41. A method for providing to a cell, tissue or organ an angiogenesis-inhibitory amount of a HPRG, an H/P domain of HPRG or a pentapeptide of said H/P domain having the sequence (His,Pro)-(His,Pro)-Pro-His-Gly (SEQ ID NO:7), or a peptide multimer that includes said pentapeptide, comprising
administering to said cell tissue or organ, the expression vector of any of claims 34-36, such that the nucleic acid is taken up and expressed in said cell, tissue or organ.

42. The method of claim 41 wherein said administering is in vivo.

43. A method for providing to a cell, tissue or organ an angiogenesis-inhibitory amount of a HPRG, an H/P domain of HPRG, a pentapeptide of said H/P domain having the sequence (His,Pro)-(His,Pro)-Pro-His-Gly (SEQ ID NO:7), or a peptide multimer that includes said pentapeptide, comprising
contacting said cell tissue or organ, with the transformed or transfected cells of any of claims 37-40, wherein said administered cells express the polypeptide, peptide or peptide multimer.

44. The method of claim 43 wherein said contacting is in vivo.

45. A method for inhibiting angiogenesis in a subject in need of such inhibition, comprising
administering to the subject an effective amount of the expression vector of any of claim 34-36, such that said nucleic acid is expressed resulting in the presence of an angiogenesis-inhibiting amount of said polypeptide, peptide or peptide multimer,
thereby inhibiting said angiogenesis.

46. A method for inhibiting angiogenesis in a subject in need of such inhibition, comprising
administering to the subject an effective amount of the transformed or transfected cells of any of claim 37-40, which cells produce and provide in the subject an angiogenesis-inhibiting amount of said polypeptide, peptide or peptide multimer,
thereby inhibiting said angiogenesis.

47. The method of claim 45 or 46 wherein said subject has a tumor, and said angiogenesis inhibition results in reduction in size or growth rate of said tumor or destruction of said tumor.

48. The method of claim 45-47 wherein said subject is a human.

49. An affinity ligand useful for binding to or isolating an HPRG-binding molecule or cells expressing the binding molecule, comprising a polypeptide, peptide or peptide multimer according to any of claims 1-4, immobilized to a solid support or carrier.

50. A method for isolating a HPRG-binding molecule from a complex mixture comprising:
(a) contacting the mixture with the affinity ligand of claim 49;
(b) allowing any material in the mixture to bind to the ligand;
(c) removing unbound material from the ligand; and
(d) eluting the bound HPRG-binding molecule.

51. A method for isolating or enriching cells expressing a HPRG-binding site or receptor from a cell mixture, comprising
(a) contacting the cell mixture with the affinity ligand of claim 49;
(b) allowing any cells expressing the binding site or receptor to bind to the affinity ligand;
(c) separating cells bound to the compound from unbound cells; and
(d) removing the bound cells, thereby isolating or enriching the HPRG binding site-expressing cells.
